(19) United States
(12) Patent Application Publication
(10) Pub. No.: US 2025/0352073 A1
(43) Pub. Date: Nov. 20, 2025

(54) SENSOR, SYSTEM AND METHOD FOR NON-CONTACT SENSING OF A PHYSIOLOGICAL PARAMETER OF A BODY

(71) Applicant: National University of Singapore, Singapore (SG)

(72) Inventors: Thanh Dat NGUYEN, Singapore (SG); Qihang ZENG, Singapore (SG); Xi TIAN, Singapore (SG); S.Y. John HO, Singapore (SG)

(21) Appl. No.: 18/863,218

(22) PCT Filed: May 12, 2023
(86) PCT No.: PCT/SG2023/050327

§ 371 Date: Nov. 5, 2024

(30) Foreign Application Priority Data
May 12, 2022 (SG) 10202204979V

(51) Int. Cl. A61B 5/0205; A61B 5/00; A61B 5/021; A61B 5/024; A61B 5/05; A61B 5/08; A61B 5/11; A61B 5/113

(52) U.S. Cl. CPC A61B 5/0205; A61B 5/05; A61B 5/1126; A61B 5/113; A61B 5/7225; A61B 5/7257; A61B 5/02125; A61B 5/02444; A61B 5/0816

(57) ABSTRACT

A sensor 104 for non-contact sensing of a physiological parameter of a body 102 is described. In an embodiment, the sensor 104 comprises: a waveguide, the waveguide comprises a metamaterial and is configured to receive a transmitted signal and to propagate the transmitted signal in a spoof surface plasmon mode along the waveguide to produce an evanescent electromagnetic field and to provide a received signal, wherein the waveguide is placed at a predetermined distance away from the body 102 for non-contact sensing of a perturbation produced by a physiological motion of the body 102 using the evanescent electromagnetic field, the perturbation produces a phase shift between the transmitted signal and the received signal for use in determining the physiological parameter of the body 102. A system 100 and a method 200 for non-contact sensing of a physiological parameter of a body 102 are also described.

### TECHNICAL FIELD

The present disclosure relates to a sensor, a system and a method for non-contact sensing of a physiological parameter of a body.

### BACKGROUND

Clinical sensing modalities for vital signs of a body typically require the attachment of electrodes and sensors on the body, mostly directly interfacing with the skin, which imposes constraints on mobility, and in many cases causes significant inconvenience or discomfort to users. Consequently, there has been increasing interests in the development of non-contact vital sign sensing systems that does not require skin contact.

Doppler radar techniques have received considerable interests in non-contact vital sign monitoring. Doppler radars rely on the detection of phase shifts in the reflected radiofrequency (RF) waves from a moving object as compared to the original transmitted waves toward that object. In this context, the detection of vital signs using radars relies on the fact that within each cardio-respiratory cycle, the resultant physical movements of the body surface due to the deformation of the heart and lung modulate the phase of the reflected signal, which is then measured. Since J. C. Lin's pioneering work in 1975 demonstrating an X-band Doppler radar system for respiration measurements, research efforts have produced smaller and lighter radar systems which are capable of power-efficient and highly accurate vital sign sensing. However, despite these advances, such systems rely on radiative RF waves, which limit practical applicability due to challenges in detecting vital signs in the presence of background noise generated for example by reflections from different parts of the body and/or other objects in the surrounding environment. This is more so for ambient health monitoring applications, where RF reflections across a large body area as well as motions of different body parts or other objects in the vicinity could cause background interference which are difficult to distinguish from the target signals.

It is therefore desirable to provide a system and a method for non-contact sensing of a physiological parameter (e.g. a vital sign) of a body which addresses the aforementioned problems and/or provides a useful alternative.

Furthermore, other desirable features and characteristics will become apparent from the subsequent detailed description and the appended claims, taken in conjunction with the accompanying drawings and this background of the disclosure.

### SUMMARY

Aspects of the present application relate to a sensor, a system and a method for non-contact sensing of a physiological parameter of a body.

In accordance with a first aspect, there is provided a sensor for non-contact sensing of a physiological parameter of a body, the sensor comprising: a waveguide, the waveguide comprises a metamaterial and is configured to receive a transmitted signal and to propagate the transmitted signal in a spoof surface plasmon mode along the waveguide to produce an evanescent electromagnetic field and to provide a received signal, wherein the waveguide is placed at a predetermined distance away from the body for non-contact sensing of a perturbation produced by a physiological motion of the body using the evanescent electromagnetic field, the perturbation produces a phase shift between the transmitted signal and the received signal for use in determining the physiological parameter of the body.

Thus, the described embodiment provides a sensor for non-contact sensing of a physiological parameter of a body. By having a waveguide configured to propagate a transmitted signal in a spoof surface plasmon mode along the waveguide to produce an evanescent electromagnetic field, and placed at a predetermined distance away from the body, the sensor for non-contact sensing of a perturbation produced by a physiological motion of the body can be used to determine a physiological parameter of the body. The use of evanescent electromagnetic field for sensing enhances sensing sensitivity as a result of spatial confinement of the electromagnetic wave. Further, the evanescent electromagnetic field is non-radiative and thereby minimises background noise or clutter which may otherwise be picked up by the waveguide due to random body motion and/or reflections from multiple objects in the surrounding. The non-contact sensing (e.g. through-clothes) of the physiological parameter using the present sensor also enables sensing/monitoring of physiological parameters/vital signs that is convenient and comfortable for multiple clinical and daily living settings, without the need for physical coupling the sensor to the skin of a body. The use of an evanescent field for localized measurement of a physiological parameter of the body also allows for multiplexed sensing of different parts of the body simultaneously to obtain multiple physiological signals, as will be illustrated in exemplary embodiments described below.

The waveguide may comprise a sensing layer on a sensing side of the waveguide adapted to detect the perturbation produced by the physiological motion of the body, a grounding layer on an opposite side to the sensing side, and a non-electrically conductive layer sandwiched between the sensing layer and the grounding layer, wherein the grounding layer is configured to confine the evanescent electromagnetic field to the sensing side of the waveguide.

The sensing layer may comprise a comb-shaped rectangular strip, the comb-shaped rectangular strip having an elongated base and a plurality of teeth extending along and from the elongated base, wherein adjacent teeth of the plurality of teeth is separated by a gap.

A height of the teeth measured from the elongated base may be adapted to vary a degree of wavelength confinement of the spoof surface plasmon mode.

In accordance with a second aspect, there is provided a system for non-contact sensing of a physiological parameter of a body, the system comprising one or more aforementioned sensors and a software-defined radio (SDR) system configured to provide the transmitted signal and to receive the received signal.

The SDR system may include a digital-to-analogue converter (DAC), and the SDR system may be configured to: generate a digital complex baseband signal; convert the digital complex baseband signal to form an analogue baseband signal using the DAC; modulate the analogue baseband signal with a carrier signal to provide the transmitted signal; demodulate the received signal to obtain in-phase and quadrature (IQ) components associated with the digital complex baseband signal; and digitise the obtained IQ components.

The SDR system may be configured to perform complex conjugate multiplication of the digital complex baseband signal and the digitised IQ components to determine a phase shift signal associated with the phase shift between the transmitted signal and the received signal.

The SDR system may be configured to filter the phase shift signal with a low-pass filter and to down-sample the filtered phase shift signal to form a decimated phase shift signal. A cut-off frequency of the low-pass filter may be more than 5 Hz and less than 200 Hz.

The SDR system may be adapted to arctangent demodulate and unwrap the decimated phase shift signal to obtain a time-varying phase signal associated with the phase shift between the transmitted signal and the received signal.

The physiological motion may be associated with more than one physiological parameter, the system may comprise a processor and a data storage storing computer program instructions operable to cause the processor to: process the time-varying phase signal with a bandpass filter to segregate the time-varying phase signal to individual components associated with each of the more than one physiological parameter.

Wherein the one or more sensors may include a first sensor provided at a back of the body adapted to detect a respiration signal and a heart signal associated with the body, and a second sensor provided at a wrist of the body adapted to detect a radial pulse signal associated with the body, the system may comprise a processor and a data storage storing computer program instructions operable to cause the processor to: process a first time-varying phase signal associated with the first sensor with bandpass filters to segregate the first time-varying phase signal to a time-varying respiration phase signal and a time-varying heart phase signal; and process a second time-varying phase signal associated with the second sensor with a bandpass filter to obtain a time-varying radial pulse phase signal.

The data storage may store computer program instructions operable to cause the processor to: perform fast Fourier transform on first 15 seconds of each signal segment of the time-varying respiration phase signal to estimate a respiratory period; calculate a moving-average curve by taking a mean of the time-varying respiration phase signal over a time window equivalent to two times of the respiratory period to generate each data point of the moving-average curve; calculate intercepts between the moving-average curve and the time-varying respiration phase signal; identify peaks on the time-varying respiration phase signal using the calculated intercepts, wherein each of the peaks is identified as a maximum between an intercept with a positive slope and an ensuing intercept with a negative slope; and calculate a respiratory cycle as a time duration between two adjacent peaks.

The data storage may store computer program instructions operable to cause the processor to: calculate a first time derivative waveform for each of the time-varying heart phase signal and the time-varying radial pulse phase signal; set all negative values of the first time derivative waveform for each of the time-varying heart phase signal and the time-varying radial pulse phase signal to zero to form a resultant waveform for each of the time-varying heart phase signal and the time-varying radial pulse phase signal; square the resultant waveform associated with each of the time-varying heart phase signal and the time-varying radial pulse phase signal to form a squared signal associated with each of the time-varying heart phase signal and the time-varying radial pulse phase signal; filter the squared signal using a moving-average filter with a predetermined time window to produce an integrated signal associated with each of the time-varying heart phase signal and the time-varying radial pulse phase signal; detect peaks in the integrated signal associated with each of the time-varying heart phase signal and the time-varying radial pulse phase signal; detect peaks in the time-varying heart phase signal and the time-varying radial pulse phase signal; verify detected peaks in the time-varying heart phase signal and the time-varying radial pulse phase signal using the detected peaks in the integrated signal; calculate beat locations in the time-varying heart phase signal and the time-varying radial pulse phase signal, each of the beat locations being a nearest preceding positive zero-intercept in relation to each verified peak of the time-varying heart phase signal and the radial pulse phase signal; and calculate beat to beat intervals associated with each of the time-varying heart phase signal and the time-varying radial pulse phase signal, the beat to beat intervals being a time interval between successive beat locations, wherein the beat to beat intervals associated with the time-varying heart phase signal relates to a heart rate and the beat to beat intervals associated with the time-varying radial pulse phase signal relates to a radial pulse rate.

The data storage may store computer program instructions operable to cause the processor to: receive the time-varying heart phase signal and the time-varying radial pulse phase signal; process the time-varying heart phase signal and the time-varying radial pulse phase signal to form a processed time-varying heart phase signal and a processed time-varying radial pulse phase signal; generate, using a trained machine learning model, an aligned time-varying heart phase signal and an aligned time-varying radial pulse phase signal based on the processed time-varying heart phase signal and the processed time-varying radial pulse phase signal, wherein peaks of the aligned time-varying heart phase signal correspond to electrocardiography (ECG) R-wave peaks and peaks of the aligned time-varying radial pulse phase signal corresponds to photoplethysmography (PPG) maximum first derivative (MFD) points; calculate a pulse transit time (PTT) as a time delay between one of the peaks of the aligned time-varying heart phase signal and a corresponding one of the peaks of the aligned time-varying radial pulse phase signal; and convert the calculated PTT to a systolic blood pressure value and a diastolic blood pressure value. Converting the calculated PTT to a systolic blood pressure value and a diastolic blood pressure value may include using a linear PTT-blood pressure relationship.

The data storage may store computer program instructions operable to cause the processor to: search, within an ensuing time window of 0.15 s to 0.4 s of the one of the peaks of the aligned time-varying heart phase signal, a local maximum of the aligned time-varying radial pulse phase signal, the local maximum being the corresponding one of the peaks for use in calculating the PTT.

The data storage may store computer program instructions operable to cause the processor to: receive training data comprising training time-varying heart phase signals and training time-varying radial pulse phase signals; process the training data to form training processed time-varying heart phase signals and training processed time-varying radial pulse phase signals; and train a machine learning model to form the trained machine learning model, wherein the data storage storing computer program instructions operable to cause the processor to train the machine learning model may store computer program instructions operable to cause the processor to: generate, using the machine learning model, training time-varying heart phase signal outputs and training time-varying radial pulse phase signal outputs based on the training processed time-varying heart phase signals and the training processed time-varying radial pulse phase signals; and minimise, using a regression layer, a mean squared error (MSE) between each of the training time-varying heart phase signal outputs and training time-varying radial pulse phase signal outputs and corresponding target time-varying heart phase signals and time-varying radial pulse phase signals for forming the trained machine learning model.

The trained machine learning model may include a long short-term memory (LSTM) network followed by a fully connected (FC) layer for each of the time-varying heart phase signal and the time-varying radial pulse phase signal.

The data storage storing computer program instructions operable to cause the processor to process the time-varying heart phase signal and the time-varying radial pulse phase signal may store computer program instructions operable to cause the processor to: left-shift the time-varying heart phase signal by a predetermined amount of time to form the processed time-varying heart phase signal; and differentiate the time-varying radial pulse phase signal with respect to time to generate a time derivative of the time-varying radial pulse phase signal to form the processed time-varying radial pulse phase signal.

The data storage may store computer program instructions operable to cause the processor to: identify epochs for blood pressure sensing, the identified epochs each being a time window having a predetermined time period during which both the time-varying heart phase signal and the time-varying radial pulse phase signal are present; calculate a mean heart rate and a mean pulse rate for the identified epochs; select, one or more candidate epoch among the identified epochs, wherein an absolute difference between the mean heart rate and the mean pulse rate of each of the one or more candidate epoch is less than two beats per minute; calculate a signal quality metric (Qₑ) for each of the time-varying heart phase signal and the time-varying radial pulse phase signal in each of the one or more candidate epoch as:

$$Qe = \frac{\frac{N}{{t}_{e}}}{\frac{1}{N - 1} {\sum }_{t = 1}^{N - 1} \frac{60}{I ( i )}}$$

where N is a number of detected beats of the time-varying heart signal or the time-varying radial pulse phase signal in each of the one or more candidate epoch, tₑ is a length of each of the corresponding one or more candidate epoch in minutes, and I is a beat-to-beat interval from each successive pair of the detected beats in seconds; and selecting one or more detection epoch from among the one or more candidate epoch for continuous blood pressure detection, wherein the signal quality metric (Qₑ) for each of the time-varying heart phase signal and the time-varying radial pulse phase signal in the one or more detection epoch is more than 0.5.

In accordance with a third aspect, there is provided a method for non-contact sensing of a physiological parameter of a body using one or more sensors, wherein each of the one or more sensors comprises a waveguide, the waveguide comprises a metamaterial and is configured to propagate a transmitted signal in a spoof surface plasmon mode along the waveguide to produce an evanescent electromagnetic field and to provide a received signal, the evanescent electromagnetic field being used for non-contact sensing of a perturbation produced by a physiological motion of the body, the method comprising: (i) placing the waveguide at a predetermined distance away from the body for non-contact sensing of the perturbation; (ii) providing the transmitted signal to the waveguide; (iii) receiving the received signal from the waveguide; and (iv) processing the received signal and the transmitted signal to determine a phase shift between the transmitted signal and the received signal caused by the perturbation for determining the physiological parameter of the body.

The waveguide may be made of a flexible material. This allows easy implementation of the waveguide in e.g. clothing for sensing or monitoring of the physiological parameter of the body.

The waveguide may comprise a sensing layer on a sensing side of the waveguide adapted to detect the perturbation produced by the physiological motion of the body, a grounding layer on an opposite side to the sensing side, and a non-electrically conductive layer sandwiched between the sensing layer and the grounding layer, wherein the grounding layer may be configured to confine the evanescent electromagnetic field to the sensing side of the waveguide. This reduces measurement noise picked up by the waveguide and improves a sensitivity of the waveguide.

The sensing layer may comprise a comb-shaped rectangular strip, the comb-shaped rectangular strip having an elongated base and a plurality of teeth extending along and from the elongated base, wherein adjacent teeth of the plurality of teeth is separated by a gap.

The method may comprise providing the transmitted signal having a frequency range of 400 MHz to 200 GHz. In embodiments, the carrier signal of the transmitted signal may have a frequency range of 400 MHz to 200 GHz.

The method may comprise: generating a digital complex baseband signal; converting the digital complex baseband signal to form an analogue baseband signal using a digital-to-analogue converter (DAC); modulating the analogue baseband signal with a carrier signal to provide the transmitted signal; demodulating the received signal to obtain in-phase and quadrature (IQ) components associated with the digital complex baseband signal; and digitizing the obtained IQ components.

The method may comprise: performing complex conjugate multiplication of the digital complex baseband signal and the digitised IQ components; and determining a phase shift signal associated with the phase shift between the transmitted signal and the received signal.

The method may comprise: filtering the phase shift signal with a low-pass filter; and down-sampling the filtered phase shift signal to form a decimated phase shift signal.

The method may comprise arctangent demodulating and unwrapping the decimated phase shift signal to obtain a time-varying phase signal associated with the phase shift between the transmitted signal and the received signal.

Wherein the one or more sensors may include a first sensor provided at a back of the body adapted to detect a respiration signal and a heart signal associated with the body, and a second sensor provided at a wrist of the body adapted to detect a radial pulse signal associated with the body, the method may comprise: processing a first time-varying phase signal associated with the first sensor with bandpass filters to segregate the first time-varying phase signal to a time-varying respiration phase signal and a time-varying heart phase signal, and processing a second time-varying phase signal associated with the second sensor with a bandpass filter to obtain a time-varying radial pulse phase signal.

The method may comprise: receiving the time-varying heart phase signal and the time-varying radial pulse phase signal; processing the time-varying heart phase signal and the time-varying radial pulse phase signal to form processed time-varying heart phase signal and processed time-varying radial pulse phase signal; generating, using a trained machine learning model, aligned time-varying heart phase signal and aligned time-varying radial pulse phase signal based on the processed time-varying heart phase signal and the processed time-varying radial pulse phase signal, wherein peaks of the aligned time-varying heart phase signal correspond to electrocardiography (ECG) R-wave peaks and peaks of the aligned time-varying radial pulse phase signal corresponds to photoplethysmography (PPG) maximum first derivative (MFD) points; calculating a pulse transit time (PTT) as a time delay between one of the peaks of the aligned time-varying heart phase signal and a corresponding one of the peaks of the aligned time-varying radial pulse phase signal; and converting the calculated PTT to a systolic blood pressure value and a diastolic blood pressure value. Converting the calculated PTT to a systolic blood pressure value and a diastolic blood pressure value may include using a linear PTT-blood pressure relationship.

The method may comprise searching, within an ensuing time window of 0.15 s to 0.4 s of the one of the peaks of the aligned time-varying heart phase signal, a local maximum of the aligned time-varying radial pulse phase signal, the local maximum being the corresponding one of the peaks for use in calculating the PTT.

The method may comprise: identifying epochs for blood pressure sensing, the identified epochs each being a time window having a predetermined time period during which both the time-varying heart phase signal and the time-varying radial pulse signal are present; calculating a mean heart rate and a mean pulse rate for the identified epochs; selecting one or more candidate epoch among the identified epochs, wherein an absolute difference between the mean heart rate and the mean pulse rate of each of the one or more candidate epoch is less than two beats per minute; calculating a signal quality metric (Qₑ) for each of the time-varying heart phase signal and the time-varying radial pulse phase signal in each of the one or more candidate epoch as:

$$Qe = \frac{\frac{N}{{t}_{e}}}{\frac{1}{N - 1} {\sum }_{t = 1}^{N - 1} \frac{60}{I ( i )}}$$

where N is a number of detected beats of the time-varying heart phase signal or the time-varying radial pulse phase signal in each of the one or more candidate epoch, tₑ is a length of each of the corresponding one or more candidate epoch in minutes, and I is a beat-to-beat interval from each successive pair of the detected beats in seconds; and selecting one or more of detection epoch from among the one or more candidate epoch for continuous blood pressure detection, wherein the signal quality metric (Qₑ) for each of the time-varying heart phase signal and the time-varying radial pulse phase signal in the one or more candidate epoch is more than 0.5.

Embodiments therefore provide a sensor, a system and a method for non-contact sensing of a physiological parameter of a body. By using a sensor comprising a waveguide configured to propagate a transmitted signal in a surface plasmon mode along the waveguide to produce an evanescent electromagnetic field, and having it placed at a predetermined distance away from the body, the sensor provides non-contact sensing of a perturbation produced by a physiological motion of the body for determining a physiological parameter of the body. The use of an evanescent electromagnetic field for sensing enhances sensing sensitivity as a result of the spatial confinement of the electromagnetic energy in the evanescent electromagnetic field. Further, the evanescent electromagnetic field is non-radiative and thereby minimizes background noise or clutter which may otherwise be picked up by the waveguide as a result of random body motion and/or reflections from multiple objects in the surrounding. The non-contact sensing (e.g. through-clothes) of the physiological parameter using the present system and method also enable sensing/monitoring of physiological parameters/vital signs that is convenient and comfortable for multiple clinical and daily living settings. The use of non-radiative localised sensing by the evanescent electromagnetic field allows for multiplexed sensing of different parts of the body simultaneously to obtain multiple physiological signals, for example a respiration rate, a heart rate and a radial pulse rate. In an embodiment, cuffless blood pressure monitoring can be achieved using the heart rate and the radial pulse rate obtained from a back and a wrist of the body, respectively, to obtain ambient vital sign monitoring.

### BRIEF DESCRIPTION OF THE DRAWINGS

Embodiments will now be described, by way of example only, with reference to the following drawings, in which:

FIGS. 1A and 1B show diagrams of a system for non-contact sensing of a physiological parameter of a body and the body in accordance with an embodiment, where FIG. 1A shows a schematic diagram of the system comprising a sensor, a software-defined radio system and a computer and FIG. 1B shows a diagram to illustrate non-contact sensing based on the sensor of FIG. 1A;

FIG. 2 is a flowchart showing steps of a method for non-contact sensing of a physiological parameter of a body using the system of FIG. 1A in accordance with an embodiment;

FIGS. 3A and 3B show images of a part of the system of FIG. 1A in accordance with an embodiment, where FIG. 3A shows an image illustrating an integration of a sensor comprising a spoof surface plasmon (SSP) metamaterial waveguide with a software-defined radio (SDR) system via coaxial cables (the SDR system is not shown) and FIG. 3B shows an image illustrating use of the system of FIG. 3A as a seat-integrated health monitoring system where heartbeat and respiration signals can be measured from a back of a body;

FIGS. 4A and 4B show diagrams of SSP waveguide simulation results in accordance with an embodiment, where FIG. 4A shows a diagram illustrating simulated |S₂₁| results without and with the presence of a human body and FIG. 4B shows a diagram illustrating an electric field of the waveguide in the xz plane when interfaced with a transmitting signal to show an interaction between the electric field and the human body for sensing (the white arrow indicates a direction of wave propagation);

FIG. 5 shows a schematic diagram to illustrate integration of a metamaterial textile sensor with a software-defined radio (SDR) system in accordance with an embodiment;

FIG. 6 shows a flowchart of a method for processing a physiological signal to detect heart beat and pulse beat in accordance with an embodiment;

FIGS. 7A and 7B show graphs of measured vital-sign waveforms in accordance with an embodiment, where FIG. 7A shows a graph of a respiration signal and FIG. 7B shows a graph of a heartbeat signal with detected beat locations (shown as circles);

FIGS. 8A, 8B and 8C show graphs for comparing heartbeats measured using the SSP metamaterial sensor of FIG. 3A and measured using a reference electrocardiogram (ECG) in accordance with an embodiment, where FIG. 8A shows a graph of measured beat-to-beat intervals (BBIs) in comparison with a reference electrocardiogram (ECG) R-peak intervals in milliseconds, FIG. 8B shows a graph of linear correlation between the BBIs measured using the SSP metamaterial sensor and the ECG measured RR intervals, and FIG. 8C shows a graph of Bland-Altman plot demonstrating a pairwise comparison between the ECG RR intervals and the SSP BBIs;

FIG. 9 shows a diagram to illustrate multi-point tracking of physiological signals from different body parts using the system of FIG. 1A in accordance with an embodiment;

FIGS. 10A and 10B show diagrams of full wave simulations of electric fields at 2.4 GHz for sensing of the radial artery in accordance with an embodiment, where FIG. 10A shows a diagram of full wave simulation of electric fields using conventional radiative sensing and FIG. 10B shows a diagram of full wave simulation of electric fields using non-radiating SSP surface waves sensing;

FIG. 11 shows an illustration of an arm placed over a metamaterial textile waveguide in accordance with an embodiment;

FIGS. 12A and 12B show graphs in relation to a geometrical parameter h of a metamaterial textile waveguide in accordance with an embodiment, where FIG. 12A shows a graph of plasmon wavenumber βₚ as a function of the geometrical parameter h for different fabric substrates and FIG. 12B shows a graph of transmission loss as a function of the textile conductivity at 2.4 GHz for different geometrical parameters h;

FIG. 13 shows a dispersion graph for metamaterial textile waveguides with varying geometrical parameter h in accordance with an embodiment;

FIGS. 14A and 14B show diagrams to illustrate a design for planar matching sections of a metamaterial textile waveguide in accordance with an embodiment, where FIG. 14A shows a diagram for illustrating a structure of the planar matching sections and FIG. 14B shows a plot of transmission coefficient versus the geometrical parameter h for varying number of matching units, N;

FIGS. 15A and 15B show diagrams of full wave simulations during sensing in accordance with embodiments, where FIG. 15A shows a diagram of a full wave simulation of a cross-section of an arm during radial pulse sensing and FIG. 15B shows a diagram of a full wave simulation of a cross-section of a human body torso during heartbeat sensing;

FIG. 16 shows a diagram of electric field profiles in the y-z plane (top) and x-z plane (bottom) of a conventional microstrip line sensor and a metamaterial textile sensor of the present disclosure in accordance with an embodiment;

FIGS. 17A and 17B show graphs of simulated |S₂₁| transmission spectra without an interaction with a body and due to interactions with different body parts in accordance with embodiments, where FIG. 17A shows a graph of the simulated |S₂₁| transmission spectra for a SSP metamaterial textile sensor and FIG. 17B shows a graph of the simulated |S₂₁| transmission spectra for a microstrip line textile sensor;

FIGS. 18A and 18B show graphs of simulated phase variations Δϕ obtained from a conventional microstrip line sensor and a metamaterial textile sensor of the present disclosure in accordance with an embodiment, where FIG. 18A shows a plot of simulated phase variations Δϕ as a result of a radial pulse within a cardiac cycle and FIG. 18B shows a plot of simulated phase variations Δϕ as a result of a heartbeat within a cardiac cycle;

FIG. 19 shows a graph of simulated phase variations Δϕ as a function of separation distance d between a conventional microstrip line sensor and a metamaterial textile sensor of the present disclosure at a back and a wrist of a body in accordance with an embodiment;

FIGS. 20A and 20B show graphs of transmission loss of the SSP metamaterial textile sensor as a function of bending radius in accordance with embodiments, where FIG. 20A shows a graph of transmission loss of the SSP metamaterial textile sensor for bending in the x-z plane and FIG. 20B shows a graph transmission loss of the SSP metamaterial textile sensor for bending in the x-y plane;

FIGS. 21A and 21B show diagrams of two SSP metamaterial textile sensor designs in accordance with embodiments, where FIG. 21A shows a U-shape sensor design for use on a chair and FIG. 21B shows a straight sensor design for use on a table;

FIG. 22 shows an illustration of a vital sign data collection setup using the system of FIG. 1A in accordance with an embodiment;

FIG. 23 shows plots of sample data collected from the two sensor channels, CH1 and CH2, using the vital sign data collection setup of FIG. 23 in accordance with an embodiment;

FIGS. 24A, 24B and 24C show Bland-Altman plots of beat-to-beat intervals obtained using reference measurements and measurements from a metamaterial textile sensor of the present disclosure in accordance with an embodiment, where FIG. 24A shows a Bland-Altman plot for respiration rate, FIG. 24B shows a Bland-Altman plot for heart rate and FIG. 24C shows a Bland-Altman plot for radial pulse rate;

FIG. 25 shows a diagram illustrating a machine learning model comprising long short-term memory (LSTM) networks and fully-connected (FC) layers for performing heart (CH1) and pulse (CH2) signal alignment to obtain pulse transit time (PTT) for blood pressure measurements in accordance with an embodiment;

FIG. 26 shows a Bland-Altman plot of the estimated PTT obtained from testing data and obtained from data using a metamaterial textile sensor of the sensing system in accordance with an embodiment;

FIG. 27 is a flowchart showing steps of a computer-implemented method for generating aligned time-varying heart phase signal and aligned time-varying radial pulse phase signal for calculating a pulse transit time (PTT) for converting to systolic and diastolic blood pressure values in accordance with an embodiment;

FIG. 28 shows a diagram illustrating an architecture and training of one LSTM aligner channel of the machine learning model of FIG. 25 in accordance with an embodiment;

FIGS. 29A and 29B show graphs of sample data for validating the machine learning model of FIG. 25 for pulse signal alignment and heart signal alignment in accordance with an embodiment, where FIG. 29A shows a graph of average detection errors against Subject ID for pulse signal alignment and FIG. 29B shows a graph of average detection errors against Subject ID for heart signal alignment;

FIG. 30 shows photographs of a multiplexed health monitoring in an office environment using a dual-channel metamaterial textile sensor system in accordance with an embodiment;

FIG. 31 shows graphs of normalized outputs from both channels CH1 and CH2, and the detected vital signs including heart rate (HR), respiratory rate (RR), systolic blood pressure (SBP) and diastolic blood pressure (DBP) in accordance with an embodiment;

FIGS. 32A and 32B show graphs of linear regression for estimating blood pressures using calculated PTT values in accordance with an embodiment, where FIG. 32A shows a graph of linear regression for estimating systolic blood pressure and FIG. 32B shows a graph of linear regression for estimating diastolic blood pressure;

FIGS. 33A and 33B show graphs in relation to selecting candidate epoch for continuous blood pressure detection in accordance with an embodiment, where FIG. 33A shows a graph for identifying candidates epoch for blood pressure sensing using an absolute difference between a mean heart rate (HR) and a mean pulse rate (PR) for each epoch, and FIG. 33B shows a graph for selecting candidate epochs identified in FIG. 33A for continuous blood pressure detection using a signal quality metric (Qₑ);

FIG. 34 shows graphs of long-term health monitoring data for normalized outputs from both channels CH1 and CH2, and the detected vital signs including heart rate (HR), respiratory rate (RR), systolic blood pressure (SBP) and diastolic blood pressure (DBP) in accordance with an embodiment;

FIG. 35 shows photographs of a passenger health monitoring system comprising a metamaterial textile sensor in accordance with an embodiment;

FIG. 36 shows graphs of normalized output and detected heart rate measured using the passenger health monitoring system of FIG. 35 and a graph of reference heart rate measured using an Apple® Watch in accordance with an embodiment; and

FIG. 37 is a block diagram showing a technical architecture of the computer of FIG. 1A in accordance with an embodiment.

### DETAILED DESCRIPTION

An exemplary embodiment relating to a sensor, a system and a method for non-contact sensing of a physiological parameter of a body is described.

Seamlessly embedded contactless sensors in ordinary physical spaces can incorporate smart sensing capabilities into otherwise passive environments. Such ambient sensing capabilities have tremendous potential to realize preventive and personalized healthcare through the unobtrusive collection of longitudinal health data during daily activities. Although microwave radars have been used as contactless physiological sensors, existing implementations are unsuitable for long-term ambient sensing due to challenges associated with sporadic interference in the complex real-world environment. In the present disclosure, contactless sensing using near-field electromagnetic interactions with the human body is described. The sensor, system and method of the present disclosure can be used for unobtrusive long-term health tracking that is immune to distant interference sources in the background. In embodiments, this provides multi-point sensing of physiological signals in ambient environments.

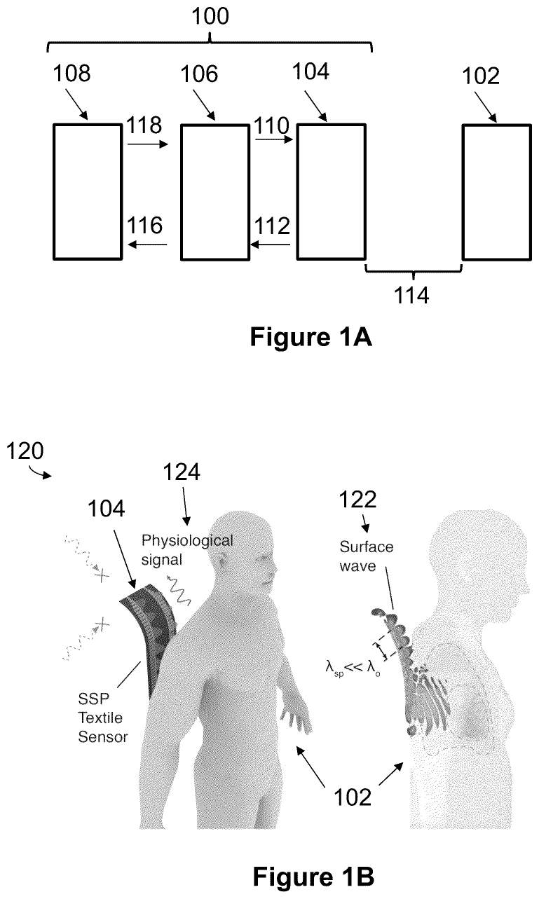

FIGS. 1A and 1B show diagrams in relation to a system 100 for non-contact sensing of a physiological parameter of a body 102 in accordance with an embodiment.

FIG. 1A shows a schematic of the system 100 for non-contact sensing of a physiological parameter of the body 102. The system 100 comprises a sensor 104, a software-defined radio (SDR) system 106 and a computer 108.

The sensor 104 comprises a waveguide. The waveguide comprises a metamaterial and is configured to propagate a transmitted signal 110 from the SDR system 106 in a spoof surface plasmon mode along the waveguide to provide a received signal 112. The transmitted signal 110 propagates in a spoof surface plasmon mode along the waveguide to produce an evanescent electromagnetic field. The evanescent electromagnetic field is used for non-contact sensing of a perturbation produced by a physiological motion of the body 102, where the sensor 104 (or the waveguide) is placed at a predetermined distance 114 away from the body 102. The predetermined distance 114 can have a range of 1 mm to 15 mm. The perturbation sensed by the evanescent electromagnetic field produces a phase shift between the transmitted signal 110 and the received signal 112, which can be processed for use in determining the physiological parameter of the body 102.

The SDR system 106 is adapted to provide the transmitted signal 110 to the sensor 104 and receive the received signal 112 from the sensor 104. In the present embodiment, the SDR system 106 is adapted to transmit the transmitted signal 110 at an entry end of the waveguide of the sensor 104, where the transmitted signal 110 propagates along the waveguide, and to receive the received signal 112 at an exit end of the waveguide of the sensor 104. The entry end of the waveguide and the exit end of the waveguide are at different ends or ports of the waveguide in the present embodiment, but it should be appreciated that the entry end and the exit end may also be at a same port of the waveguide in other embodiments.

Once the received signal 112 is received by the SDR system 106, the transmitted signal 110 and the received signal 112 can be processed to determine the phase shift caused by the physiological motion of the body 102. The information or signals in relation to the determined phase shift can then be used to determine a physiological parameter of the body 102 associated with the physiological motion. Although a SDR system 106 is used in the present embodiment for providing the transmitted signal 110 and for receiving the received signal 112, a skilled person would appreciate that this is not to be construed as limiting, and that other suitable signal system (e.g. a radio-frequency transceiver integrated on a chip) may be used as long as the phase shift caused by the physiological motion of the body 102 can be determined.

The computer 108 comprises a processor and a data storage storing computer program instructions operable to cause the processor to perform various processes on time-varying phase signals 116 received from the SDR system 106. For example, where the physiological motion of the body 102 is associated with more than one physiological parameter, the computer 108 may be adapted to process a time-varying phase signal with a bandpass filter to segregate the time-varying phase signal to individual components associated with each of the more than one physiological parameter. The computer 108 can be further configured to pre-process the time-varying phase signals 116, calculate a respiratory cycle and calculate beat-to-beat intervals for obtaining a heart rate and a radial pulse rate using the received time-varying phase signals 116. In an embodiment, the data storage of the computer 108 stores a trained machine learning model to generate aligned time-varying heart phase signal and aligned time-varying radial pulse phase signal for calculating a pulse transit time (PTT) and to convert the calculated PTT to a systolic blood pressure value and a diastolic blood pressure value. Other processes which can be performed by the computer 108 are described in relation to subsequent Figures.

In an embodiment, the computer 108 can be used to provide instructions 118 to the SDR system 106, for example, in relation to controlling parameters associated with the transmitted signal 110 and/or controlling the processing parameters of the received signal 112. In another embodiment, the SDR system 106 can be operated independently from the computer 108. For example, instructions for waveform generation and signal processing can be programmed into the SDR system 106 as an embedded application. In this case, the SDR system 106 is used to provide the time-varying phase signals 116 to the computer 108 for processing.

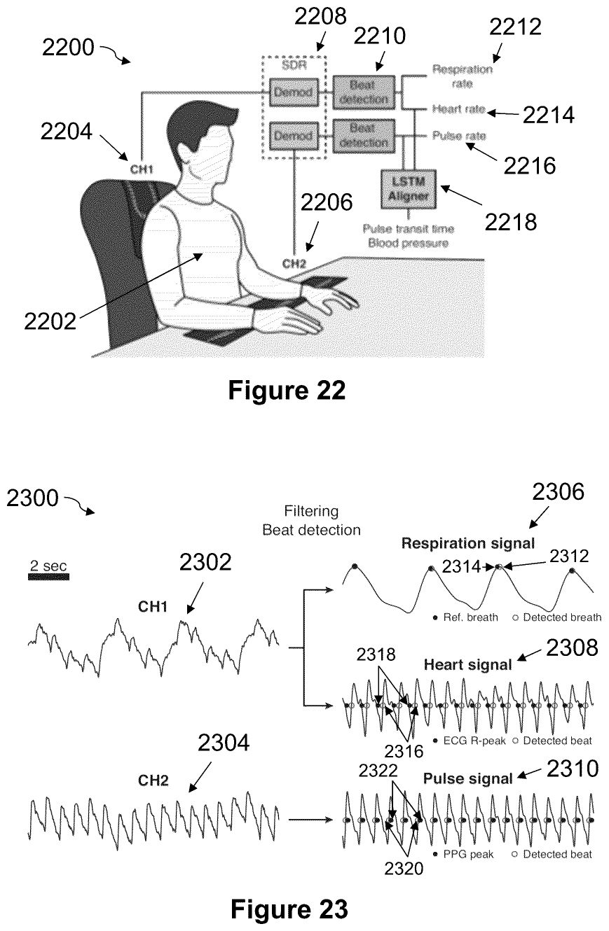

Although there is only one sensor 104 shown in the system 100 of FIG. 1A, it should be appreciated that more than one sensor 104 can be operationally connected to the SDR system 106 and the computer 108. For example, as described in relation to FIG. 22, a sensor can be provided on a table for non-contacting sensing of a radial pulse from wrists of the body 102 and a sensor can be provided on a chair for non-contacting sensing of a heart pulse and a respiratory rate of the body 102. Further, the SDR system 106 and the computer 108 are shown as separate entities in FIG. 1A, but it should be appreciated that in an embodiment, the SDR system 106 can be part of the computer 108 or that the SDR system and the computer 104 can be integrated to form a single processing unit. For example, an application-specific device can be engineered to integrate the RF functionalities of the SDR system 106 with computing functionalities required to operate it.

FIG. 1B shows a diagram 120 to illustrate non-contact sensing based on the sensor 104 of FIG. 1A.

The principle of ambient near-field health sensing using the sensor 104 is illustrated in FIG. 1B. In the present embodiment, the sensor 104 is a metamaterial textile sensor designed to propagate electromagnetic surface waves 122 in the spoof-surface-plasmonic (SSP) modes, which extend evanescently in the normal direction to its surface. Due to the non-radiating nature of the surface waves 122, the sensor 104 is unaffected by distant interference sources in the environment such as background clutter or motions of other people, while facilitating sensitive close-range interactions with a part or parts of the body 102. In particular, sensitivity to subtle physiological motions or signals 124 is enhanced by the dual action of the shortened surface plasmon wavelength (λₛₚ) relative to free-space wavelength (λ₀) and the confinement of electromagnetic energy on the sensing surface, which can strongly couple with the heterogeneous tissue layers of the body 102 and respond to cardiopulmonary-mediated impedance modulation. Particularly, the immunity to distant interference sources is brought about by the non-radiating and localized nature of SSP surface waves. The sub-wavelength energy confinement on the sensor leads to an enhanced coupling with tissues. The shortened surface plasmon wavelength (λₛₚ«λ_{D}) results in amplified Doppler phase variations from cardiopulmonary-mediated impedance modulations.

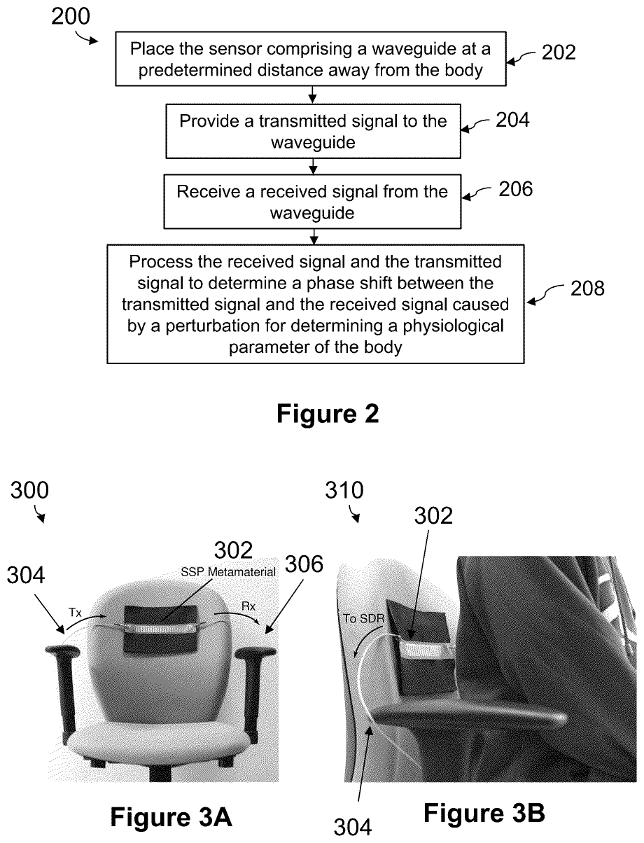

FIG. 2 is a flowchart showing steps of a method 200 for non-contact sensing of a physiological parameter of a body 102 using the system 100 of FIG. 1 in accordance with an embodiment. The method uses the sensor 104 as described above.

In a step 202, the sensor 104 is placed at a predetermined distance 114 away from the body 102 for non-contact sensing. The predetermined distance can be a range of suitable distances (e.g. 1 mm to 15 mm) away from the body 102 so it provides some flexibility. The body 102 is preferably stilled during measurements.

In a step 204, a transmitted signal 110 is provided to the sensor 104. In the present embodiment, the transmitted signal 110 is provided by the SDR system 106.

In a step 206, a received signal 112 is received from the sensor 104, after the transmitted signal 110 has been propagated through the waveguide of the sensor 104. In the present embodiment, the received signal 112 is received by the SDR system 106.

In a step 208, the received signal 112 and the transmitted signal 110 are processed to determine a phase shift between the transmitted signal 110 and the received signal 112 which is caused by the perturbation produced by physiological motion of the body 102. The determined phase shift can then be used or processed for determining the physiological parameter of the body 102. Particularly, in an embodiment, the SDR system 106 is configured to: generate a digital complex baseband signal, convert the digital complex baseband signal to form an analogue baseband signal using a digital-to-analogue converter (DAC), modulate the analogue baseband signal with a carrier signal to provide the transmitted signal, demodulate the received signal to obtain in-phase and quadrature (IQ) components associated with the digital complex baseband signal, and digitise the obtained IQ components. The SDR system 106 is then configured to perform complex conjugate multiplication of the digital complex baseband signal and the digitised IQ components to determine a phase shift signal associated with the phase shift between the transmitted signal and the received signal. The SDR system 106 is adapted to arctangent demodulate and unwrap the phase shift signal to obtain a time-varying phase signal associated with the phase shift between the transmitted signal and the received signal. The time-varying phase signal obtained from the SDR system 106 is then processed by the computer 108 to obtain, for example, a respiratory cycle, beat-to-beat intervals associated with a heart rate, and/or beat-to-beat intervals associated with a radial pulse rate. This will be further discussed in relation to subsequent Figures.

In the embodiments of the present disclosure, a class of thin, flexible waveguides that can support spoof surface plasmons (SSP) at radio frequencies was developed using metamaterials. Waveguides formed using conductive textile metamaterials can be integrated on clothing, which can significantly increase a transmission efficiency of wireless signals around a body (e.g. a human body). These waveguides exploit the evanescent field associated with SSP modes to wirelessly interact with nearby wearable devices or a body for non-contact sensing of a physiological parameter or a vital sign of the body.

A multiplexed non-contact health monitoring system (or vital sign monitoring system) was developed by integrating a sensor having a spoof surface plasmon (SSP) metamaterial waveguide with software-defined radar techniques. In the present embodiment, the SSP metamaterial is used to provide a spatially selective sensing modality for cardiopulmonary motions. The SSP metamaterial waveguides can be integrated with different household furniture or clothing, enabling simultaneous monitoring of physiological parameters (e.g. respiration rate and heart rate) in the context of daily living. The metamaterial sensor is thin and soft, and is able to monitor health through clothing, accessories or furniture.

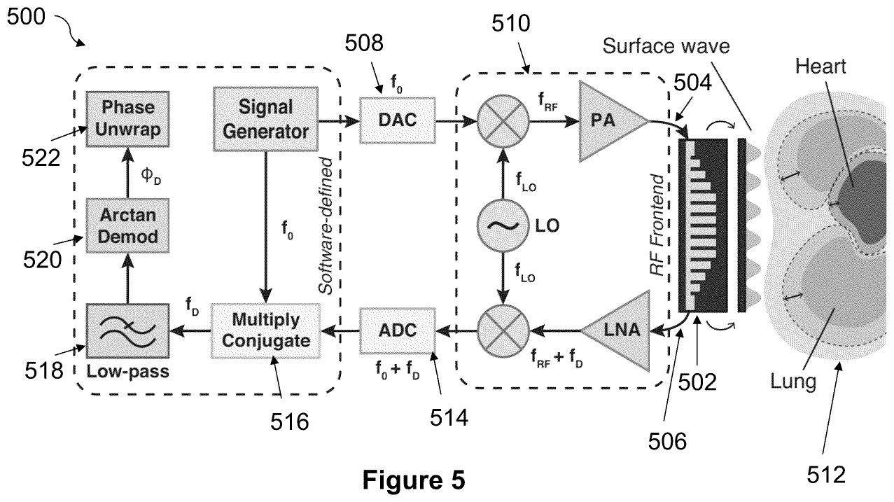

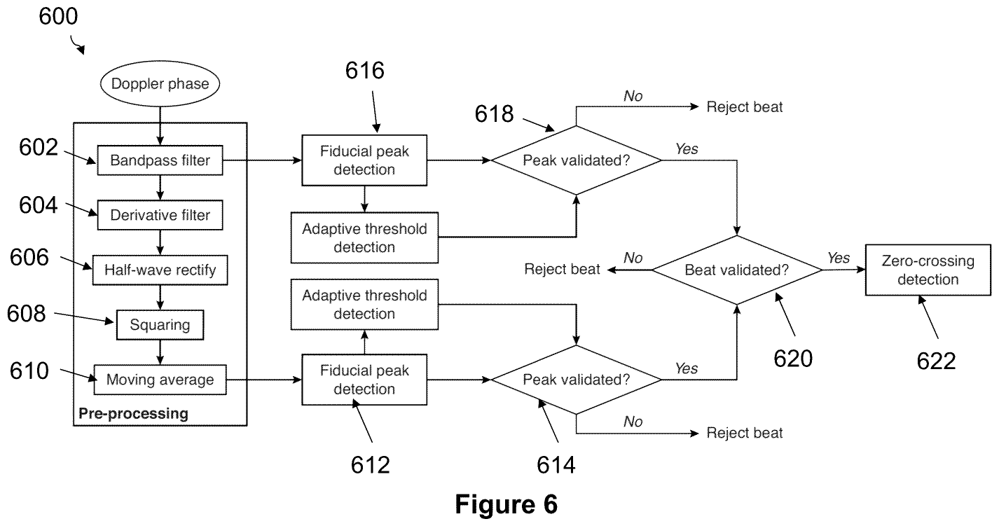

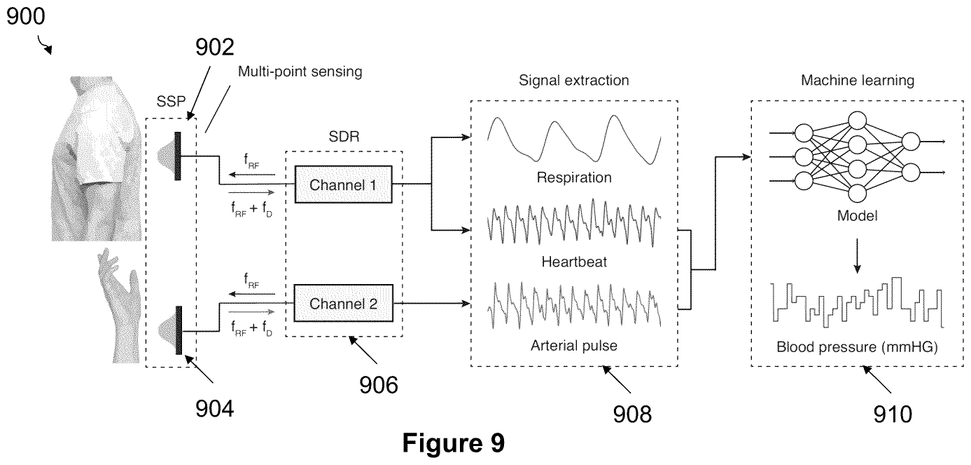

FIGS. 3A to 4B, and FIGS. 7A to 8C relate to a first embodiment of the sensor 104 used in the system 100 which was placed on a chair at the back of the body for non-contact physiological parameters sensing. FIGS. 5 and 6 relate to the SDR system 106 and a method of detection a heart beat or a radial pulse beat, respectively, and are also applied to subsequent embodiments as described in relation to FIGS. 9 to 36.

FIGS. 3A and 3B show images 300, 310 of part of the system 100 of FIG. 1 in accordance with an embodiment. As described in relation to FIG. 1A, the system 100 includes the sensor 104 comprising a SSP waveguide, and the software-defined radio (SDR) system 106 for radar signal transduction and acquisition. FIG. 3A shows the image 300 illustrating the SSP metamaterial waveguide 302 being connected with the SDR system 106 via coaxial cables 304, 306 (note that the SDR system 106 is not shown in FIG. 3A). The SSP waveguide 302 was designed to support surface plasmon-like modes of propagation at a working frequency of 2.4 GHZ, guiding a transmitted signal from a transmission port to a receiving port of the SDR system 106. In the present embodiment, as shown in FIG. 3A, the transmitted signal Tx is received at one end of the waveguide 302 and the received signal Rx is provided at an opposite end of the waveguide 302. As opposed to radiative elements, the SSP metamaterial exhibits a tight spatial wave confinement and high local field intensity in a surrounding space. Consequently, a small perturbation near the SPP waveguide 302 will lead to a disproportionate change in the electromagnetic fields at that location. This change or perturbation in the localized electromagnetic field (or evanescent electromagnetic field) can be captured as a phase shift (or a Doppler phase shift) between the transmitted signal Tx and the received signal Rx, making it highly sensitive to small physiological motion, such as those resulting from cardiopulmonary actions of a body. In this way, physiological parameters or vital signs such as respiratory rate and/or heart rate could be obtained from the back of the body in the seated position as shown in the image 310 of FIG. 3B.

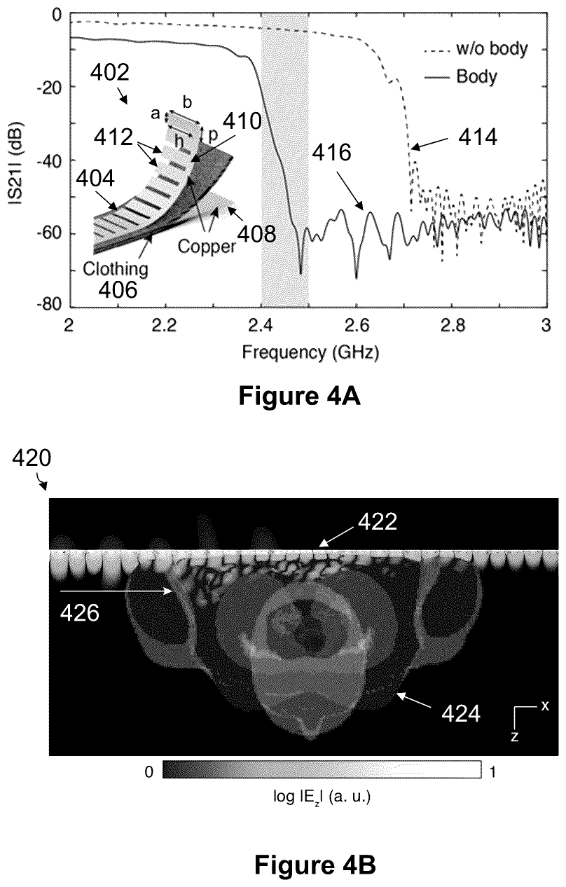

To realize the system 100 of the present embodiment, a suitable waveguide for use with the sensor 104 was designed and analysed. The waveguide 402 of the present embodiment includes a metamaterial and has a structure which includes a planar comb pattern as a top layer 404, a middle non-electrically conductive layer (e.g. a fabric layer) 406, and an unpatterned bottom layer 408. This is shown in the inset of FIG. 4A. The top layer 404 is used as a sensing layer and it comprises a comb-shaped rectangular strip having an elongated base 410 and a plurality of teeth 412 extending along and from the elongated base 410 where adjacent teeth of the plurality of teeth 412 is separated by a gap. The unpatterned bottom layer 408 acts as a grounding layer or a ground plane for confining microwave energy to only the sensing side (i.e. the side of the top layer or the sensing layer) of the waveguide 402. Parameters of the structure of the waveguide 402 was optimized by computing dispersion curves using an eigenmode solver (in the present embodiment, CST Microwave Studio). In the present embodiment, geometrical parameters of the structure were optimized to having: a height of a tooth, h=18 mm; a width of the rectangular strip of the sensing layer, b=22 mm; a width of a tooth, a=6 mm; and a length of a period (i.e. a distance between adjacent teeth), p=8 mm (i.e. with a gap (i.e. p−a) between two adjacent teeth of 2 mm).

FIG. 4A also shows plots of simulated |S₂₁| results without (i.e. plot 414) and with the presence of a human torso or human body (i.e. plot 416). The plots 414, 416 were obtained using a straight SSP waveguide design. The simulated S₂₁ comparison as shown in FIG. 4A indicates the coupling of electromagnetic energy from the waveguide 402 to the human body, as observed from a reduction in the transmission coefficient |S₂₁| at the working frequency band and the left-shifted low-pass characteristics. In the present embodiment, the sensing layer 404 and the grounding layer 408 of the SSP waveguide 402 in the were fabricated by a commercial printed circuit board (PCB) process using 35-μm thick copper on a flexible polyimide (PI) substrate.

To show the interaction between the SSP waveguide 402 and the body, a full-wave simulation of the SSP waveguide 402 placed on a voxel model of the human body (Donna, CST Microwave Studio) which includes the tissue heterogeneity of the body, was performed. Radio frequency (RF) signals were transmitted from the SDR system to the SSP waveguide 402 and propagated along the SSP waveguide 402 as surface waves.

FIG. 4B shows a diagram 420 illustrating an electric field in the xz plane in a portion of the waveguide 422 when the waveguide 422 was interfaced with a transmitting signal from the SDR system to show an interaction between the electric field and a human body 424 for sensing. The white arrow 426 of FIG. 4B indicates a direction of wave propagation of the transmitted signal. As shown in FIG. 4B, a large portion of the RF energy is coupled into the human body 424 before being provided as a received signal at an exit end of the waveguide and received at a receiver (Rx) port of the SDR system. The amount of coupling can be described by Pₜᵢₛₛᵤₑ=Pᵢₙ, where Pₜᵢₛₛᵤₑ is the power loss in biological tissues and Pᵢₙ is the input power to the SSP waveguide 422.

This was evaluated as 70.4% for a transmitted signal frequency at 2.4 GHz in the present embodiment.

To demonstrate sensing of a physiological parameter or physiological parameters using the aforementioned system 100, the SSP waveguide was interfaced with a USRP B210 SDR system (Ettus Research, National Instruments) via coaxial cables.

FIG. 5 shows a schematic diagram to illustrate integration of a textile sensor with a software-defined radio (SDR) system 500 in accordance with an embodiment.

As shown in FIG. 5, the textile sensor 502 was set up as a 2-port device, with each port or terminal of the sensor 502 being connected respectively to a Tx channel 504 and a Rx channel 506 of the SDR (Ettus B210 USRP, National Instruments) 500 with coaxial cables.

A software-defined continuous-wave signal for use as a transmitted signal f_{RF} was configured by (i) generating a digital complex baseband signal using the SDR system 500 which was passed through a built-in digital-to-analogue converter (DAC) 508 to form an analogue baseband signal f₀ and (ii) mixing or modulating the analogue baseband signal f₀ with a 2.4 GHz carrier frequency, f_{LO}, generated by the RF front end 510 of the SDR system 500. The transmitted signals f_{RF} (at 2.4 GHZ) were therefore upconverted from the f₀=1 MHz baseband signals using the RF front end 510 and were guided conformally across the textile sensor 502. The transmitted (Tx) signal (i.e. f_{RF}) was guided along the SSP waveguide of the textile sensor 502, interacting with a body 512, and collected back as a received (Rx) signal (i.e. f_{RF}+f_{D}) at the SDR system 500. Interactions with the body 512 lead to Doppler phase changes captured as f₀ mixed in the return RF signal (i.e. the received (Rx) signal). At the receiving port, the Rx signal was quadrature-demodulated to the baseband by the SDR's analogue RF front-end 510, and the resultant in-phase and quadrature (IQ) signals were digitised at a 1 MHz sampling rate. In the present embodiment, the SDR system 500 includes a built-in analogue-to-digital converter (ADC) 514 after the radio-frequency (RF) analogue front end 510 which digitises the IQ signals after demodulation. The phase shifts (or Doppler phase shifts, i.e. the f_{D} components) produced by the physiological movements in the digital domain were obtained as a phase shift signal by complex conjugate multiplication 516 of the baseband signal f_{D} and the Rx signals, followed by low pass filtering 518 and decimation to 400 Hz. This first low-pass filtering step and down-sampling step are for removing unwanted high-frequency components in the signals received by the SDR system 106 and for recording the data at a lower sampling rate to reduce file sizes in relation to these signals, respectively. The baseband Rx signals are the low-frequency components that are down-converted from the 2.4 GHz carrier frequency signal. The Doppler phase variations ϕ_{D} are obtained by arctangent demodulation 520 and then unwrapped 522 to produce the sensor outputs. This is shown in relation to Equations (1) and (2) below.

The digital complex Doppler phase shift signals were obtained by conjugate multiplication of the baseband Tx signal and the Rx signal after downconversion:

$${e}^{j 2 π {f}_{BB} t} \times {e}^{- j 2 π {f}_{BB} t + j {∅}_{D} ( t )} = {e}^{j {∅}_{D} ( t )} \qquad ( 1 )$$

where e^{jØ}^{_{D}}^{(t)} represents the captured Doppler shifts caused by physiological actions. In the present embodiments, the SDR is programmed to transmit (Tx) and receive (Rx) continuous wave (CW) signals at the carrier frequency f_{RF}=2.4 GHZ, with the baseband tone set at f_{BB}=10 KHz sampled at 1 MHz. At 10 dBm Tx power, the simulated whole body averaged specific absorption rate (SAR) is 0.019 W/kg, well below the IEEE C.95.1-2019 limit of 2 W/kg.

The time-varying Doppler phase shift ϕ_{D} (t) is then obtained by arctangent demodulation and unwrapping of the complex Doppler signal using the following:

$${∅}_{D} ( t ) = unwrap ( {tan}^{- 1} ( \frac{sin {∅}_{D} ( t )}{cos {∅}_{D} ( t )} ) ) \qquad ( 2 )$$

In the present embodiments, the outlined SDR system 500 was implemented using the open-source software GNU Radio on a personal computer. The personal computer is used to power and communicate with the SDR system over universal serial bus (USB). Controls of the SDR system 500 were performed using the GNU Radio software.

Using the setup shown in FIG. 3A, the vital signs or physiological parameters of a human subject were measured to verify a sensing capability of the system. A fully clothed subject was instructed to be seated on the configured office chair (see e.g. FIG. 3B) and to lean lightly against the backrest. The raw Doppler phase shift signal was recorded by the GNU Radio software using the configuration described above. As the Doppler phase information contains a superposition of both respiration and heartbeat, in the present embodiment, the recorded time-varying phase shift signal was processed using MATLAB on the computer 108 with bandpass filters to produce either respiration signals (or time-varying respiration phase signal) (e.g. having a frequency range of 0.1-0.8 Hz) or heartbeat signals (or time-varying heart phase signal) (e.g. having a frequency range of 0.9-5 Hz). In an embodiment where a second sensor is used to detect radial pulse signals (see for example in relation to FIG. 22 below), the computer 108 is adapted to filter a time-varying phase shift signal received from the second sensor using a bandpass filter (e.g. having a frequency range of 0.9-5 Hz) to obtain a time-varying radial pulse phase signal. The accuracy of the present system for heartbeat sensing was validated by simultaneously measuring heart rate using the present system and measuring a reference electrocardiogram (ECG) signal (MP46, BIOPAC Systems, Inc.).

In relation to detecting a respiratory cycle, a respiratory peak detection algorithm was implemented based on moving-average curve (MAC) intercepts on both the sensor data and the BIOPAC reference data. As described above, in the present embodiments, the sensor output (i.e. the time-varying phase shift signal ϕ_{D} (t)) was first bandpass filtered between 0.1 Hz and 0.8 Hz to obtain the time-varying respiration phase signal. Fast Fourier transform (FFT) was applied on the first 15 s of each time-varying respiration phase signal segment to estimate a respiratory period Tₑₛₜ. The MAC was calculated at every point by taking the mean of the time-varying respiration phase signal over a time window equivalent to 2Tₑₛₜ to generate each data point of the MAC. The intercepts between the moving-average curve and the time-varying respiration phase signal were calculated and were labelled as either up or down intercepts based on the slope of the signal at that point. A peak is identified as a maximum between an intercept with a positive slope and an ensuing intercept with a negative slope, and a respiratory cycle is calculated as the time duration between two adjacent peaks.

FIG. 6 shows a flowchart of a method 600 for processing a physiological signal to detect heart beats and pulse beats in accordance with an embodiment.

To extract heart beats or pulse beats, a zero-crossing detection algorithm was implemented with adaptive thresholds based on the Pan-Tompkins algorithm. As described above, sensor outputs (i.e. the time-varying phase shift signals ϕ_{D} (t)) in relation to heart or pulse waveforms were first bandpass filtered in a step 602 from 0.9 Hz to 5 Hz to remove direct current (DC) drifts, respiration and high-frequency noise and to obtain the time-varying heart phase signal or the time-varying radial pulse phase signal, respectively. A first time-derivative waveform was then calculated using a derivative filter in a step 604 for each of the time-varying heart phase signal and the time-varying radial pulse phase signal. The first time-derivative waveform was then half-wave rectified in a step 606 (i.e. set all negative values to zero), and the resultant waveform was squared in a step 608 to minimise small noise peaks. The squared signal was then passed through a moving-average filter, in a step 610, with 150 ms windows to produce an integrated signal used in subsequent beat detection. Peaks in the integrated signal are detected in a step 612 and in the present embodiment, validated in a step 614 using adaptive thresholding and decision rules according to the original Pan-Tompkins algorithm. Similarly, peaks in the time-varying heart phase signal and the time-varying radial pulse phase signal from the step 602 were detected in a step 616 and in the present embodiment, validated in a step 618 using adaptive thresholding and decision rules according to the original Pan-Tompkins algorithm. Detected peaks in the time-varying heart phase signal and the time-varying radial pulse phase signal are verified in a step 620 using the detected peaks in the integrated signal. Beat locations in the time-varying heart phase signal and the time-varying radial pulse phase signal are then calculated in a step 622, where each of the beat locations is a nearest preceding positive zero-intercept in relation to each verified peak of the time-varying heart phase signal and the time-varying radial pulse phase signal (i.e. outputs from the step 602).

Beat to beat intervals associated with each of the time-varying heart phase signal and the time-varying radial pulse phase signal (e.g. in relation to the heart beat waveforms or the radial pulse waveforms) can then be calculated as a time interval between successive beat locations.

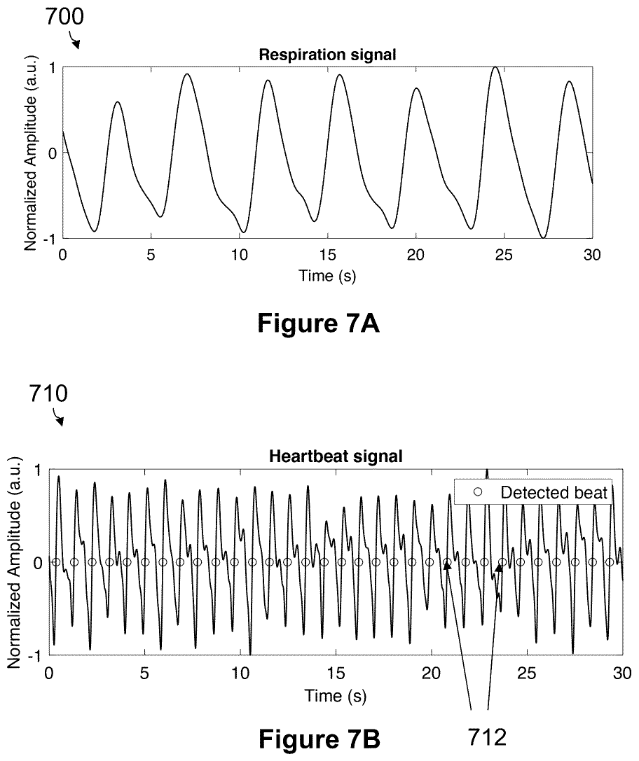

FIGS. 7A and 7B shows graphs 700, 710 of measured vital-sign waveforms in accordance with an embodiment, where FIG. 7A shows a graph 700 of a respiration signal and FIG. 7B shows a graph 710 of a heartbeat signal with detected beat locations (shown as circles 712). The normalized respiration signal (or normalized time-varying respiration phase signal) (as shown in FIG. 7A) and the normalized heartbeat signal (or normalized time-varying heart phase signal) (as shown in FIG. 7B) were obtained simultaneously from a same recording or measurements using the waveguide 302. The detected heartbeat locations using the afore-described method 600 are shown on the filtered signal as circles 712 in FIG. 7B.

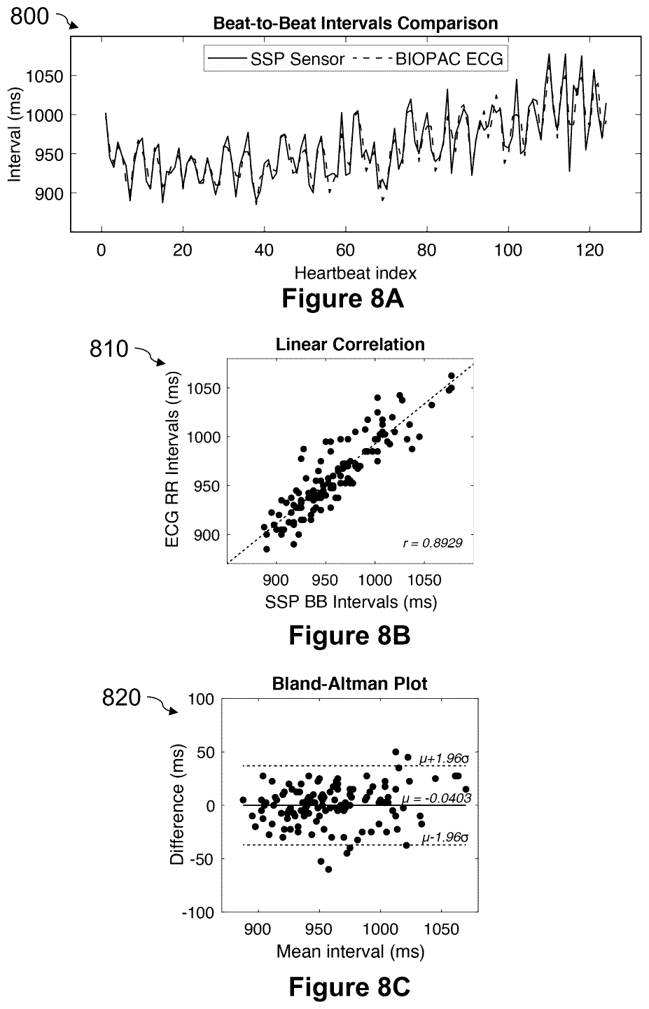

FIG. 8A shows a graph 800 of measured beat-to-beat intervals (BBIs) obtained from the SSP waveguide 302 of the sensor 104. This is being compared with reference electrocardiogram (ECG) R-peak (RR) intervals in milliseconds for 125 heartbeats using a 120-second data record in the graph 800. FIG. 8A shows a good agreement between the BBIs obtained using the present system 100 and the reference data.

FIG. 8B shows a graph 810 of linear correlation between the BBIs measured using the SSP sensor 104 and the ECG measured intervals. As shown in FIG. 8B, the BBI measurements show a strong positive linear correlation with the reference ECG RR intervals, with a Pearson correlation coefficient r=0.8929 (P<0.001).

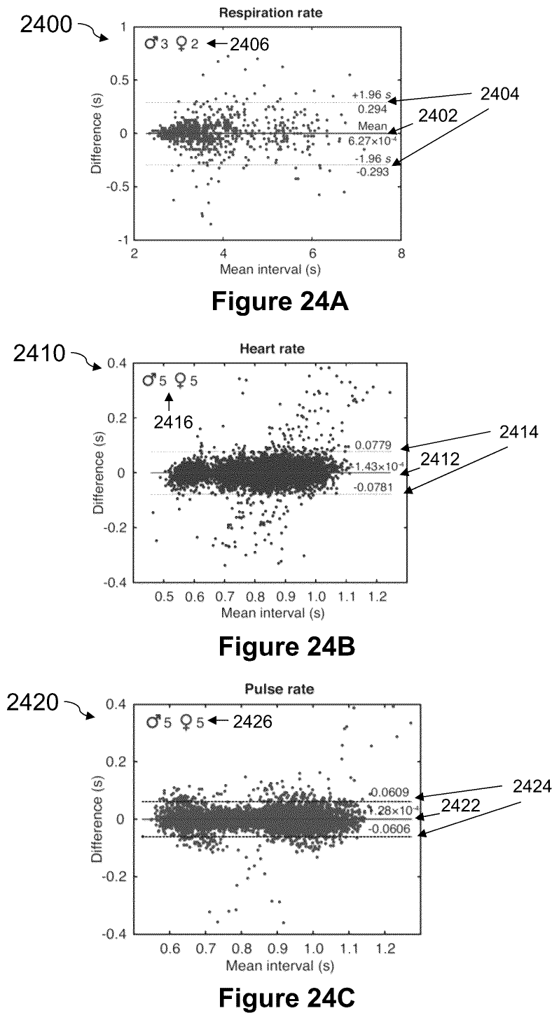

FIG. 8C shows a Bland-Altman plot 820 showing a pairwise comparison between the ECG RR intervals and the SSP BBIs, which shows a mean of difference μ=−0.0403 ms and a standard deviation σ=1.8893 ms. The Bland-Altman analysis in FIG. 8C shows an excellent agreement between the present system 100 and the ECG sensor, as demonstrated by the small bias (μ=−0.0403 ms) and more than 95% of data points lying within the limits of agreement (LoA=[μ−1.96σ; μ+1.96σ]=[−37.0634; 36.9828] ms). The Bland-Altman plot 820 as shown relates to data obtained from 1 subject among 10 subjects. The Bland-Altman plot for the 10 subjects are shown subsequently in relation to FIG. 24B.

FIG. 9 to FIG. 32B below provide another example of using the system 100 where two sensors 104 were used to track physiological signals from different parts of the body 102.

FIG. 9 shows a diagram 900 to illustrate multi-point tracking of physiological signals from different body parts using the system 100 of FIG. 1A in accordance with an embodiment.

The multi-point tracking of physiological signals from different body parts is enabled by the localized sensing property of the SSP sensor. In the present embodiment, a first SSP sensor 902 is provided at or near a back of the body and a second SSP sensor 904 is provided at or near a wrist of the body. Being thin and soft, the SSP sensors 902, 904 can be easily integrated into everyday objects for the simultaneous monitoring of multiple localized vital signs on the body, such as respiration and heartbeat signals from the back and radial pulse signal from the wrist as shown in FIG. 9.

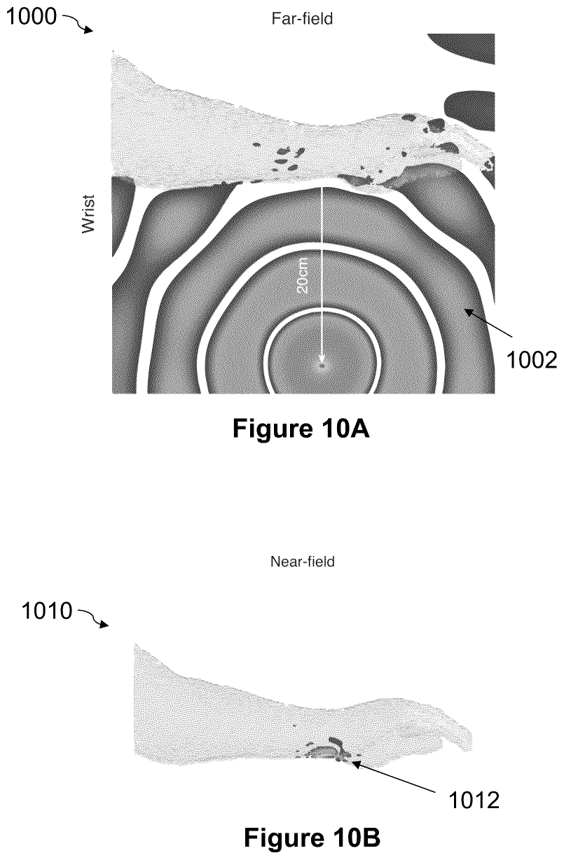

Similar to the earlier embodiment, the present embodiment relating to multi-point tracking of physiological signals include the SSP sensors 902, 904 being interfaced with the SDR system 906 being programmed to transmit a continuous wave (CW) transmitted (Tx) signal at a frequency f_{RF}=2.4 GHZ, which is propagated conformally along each of the SSP sensors 902, 904 as SSP surface waves allowing it to interact strongly with biological tissues in the close range. Consequently, small perturbations near sensing surfaces of the SSP sensors 902, 904 due to cardiopulmonary activities will lead to large corresponding changes in the electromagnetic fields at that location, which are captured as Doppler phase variations in the received (Rx) signal (i.e. f_{RF}+f_{D}) at the SDR system 906. This provides the localized sensing capability of these SSP sensors 902, 904 which allows the SSP sensors 902, 904 to obtain physiological information from smaller body parts such as the radial artery, in contrast to conventional radar sensors which detect free-space radiation and are therefore subjected to background noises over a large space. This is illustrated in relation to FIGS. 10A and 10B.

FIGS. 10A and 10B shows diagrams 1000, 1010 of full wave simulations of electric fields at 2.4 GHz for sensing of the radial artery in accordance with an embodiment. As shown in FIG. 10A, conventional radiative sensing involves detection of free-space radiation over a large area or space 1002. In contrast, as shown in FIG. 10B, the present sensing of perturbations using SSP surface waves involves non-radiating sensing over a localised area/space 1012.

Referring back to FIG. 9, processing of the received (Rx) signal is performed at the SDR system 906 (details were discussed above and so will not be repeated here for succinctness) to obtain the time-varying Doppler phase shift ϕ_{D} (t) signals at each of channels 1 and 2. The time-varying Doppler phase shift ϕ_{D} (t) signals were bandpass filtered using MATLAB (R2021a, The MathWorks, Inc.) on the computer 108 to obtain the respiration signals (also known as time-varying respiration phase signal in the present disclosure) (e.g. having a frequency range of 0.1 Hz-0.8 Hz) and the heartbeat signals (also known as time-varying heart phase signal in the present disclosure) (e.g. having a frequency range of 0.9 Hz-5 Hz) at channel 1, as well as the radial pulse signals (also known as time-varying radial pulse phase signal in the present disclosure) (e.g. having a frequency range of 0.9 Hz-5 Hz) at channel 2. This is shown at 908 of FIG. 9. The reference heart and pulse beat information were obtained directly from the BIOPAC Student Lab Software.

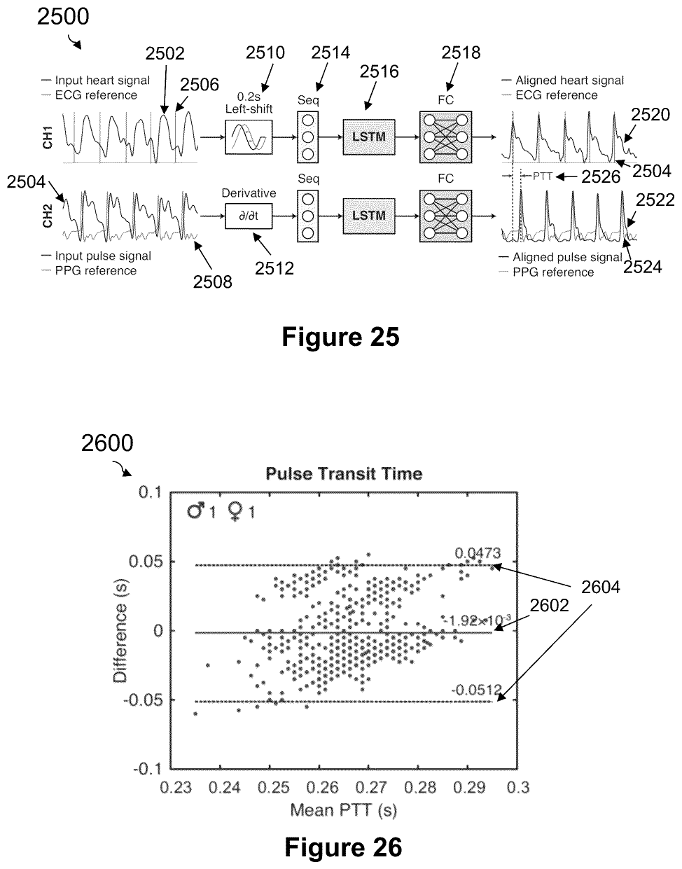

The heartbeat signals and the radial pulse signals can be further processed using a machine learning model at the computer 108 to obtain blood pressure values. This is shown at 910 of FIG. 9, and will be discussed in further details in relation to FIGS. 25 to 28 below.

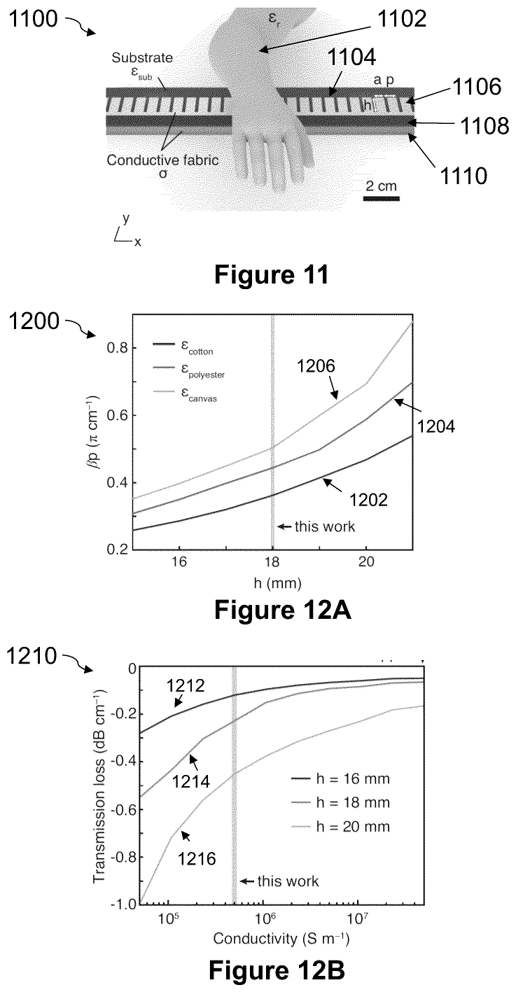

FIG. 11 shows an illustration 1100 of an arm 1102 placed over a metamaterial textile waveguide 1104 in accordance with an embodiment. The metamaterial textile waveguide 1104 comprises a patterned conductive top layer 1106, an intermediate non-conductive layer 1108 and a conductive bottom isolation layer 1110. Similar to the earlier embodiment, the patterned conductive top layer 1106 is used as a sensing layer and comprises a comb-shaped rectangular strip having an elongated base and a plurality of teeth extending along and from the elongated base where adjacent teeth of the plurality of teeth is separated by a gap. The conductive bottom isolation layer 1110 acts as a grounding layer or a ground plane for confining microwave energy to only the sensing side (i.e. the side of the top layer or the sensing layer) of the waveguide 1104.

In this embodiment, to fabricate the textile waveguide 1104, commercially available conductive textiles (Holland Shielding Systems) were patterned by laser cutting (Universal Laser Systems, VLS 2.30) and attached to polyester fabric sheets. SMA connectors were attached by adhesive coating using conductive epoxy (CW 2460, Chemtronics) at room temperature. Compared to the waveguide in an earlier embodiment as shown in relation to FIG. 4, the textile waveguide 1104 in this embodiment does not require a substrate as the conductive fabrics/textiles used in the patterned conductive top layer 1106 and the conductive bottom isolation layer 1110 can be attached directly to the polyester fabric sheet which forms the intermediate non-conductive layer 1108.

CST Microwave Studio (Dassault Systems) was used for electromagnetic simulations for the results as shown in FIGS. 12A to 20B.

FIGS. 12A and 12B show graphs 1200, 1210 in relation to a geometrical parameter h of the metamaterial textile waveguide 1104 of FIG. 11, where the parameter h relates to a height of a tooth of the patterned conductive top layer 1106.

FIG. 12A shows the graph 1200 of plasmon wavenumber βp as a function of the geometrical parameter h for different fabric substrates as the intermediate non-conductive layer 1108. The three different fabric substrates used in the present embodiment includes a cotton fabric, a polyester fabric and a canvas fabric, each having permittivity of ε_{cotton}, ε_{polyester}, and ε_{canvas}, respectively. The materials for substrate fabric were assigned with ε_{cotton}=1.6, ε_{polyester}=1.9, and ε_{canvas}=2.1. A plot 1202 is shown for the cotton fabric, a plot of 1204 is shown for the polyester fabric and a plot of 1206 is shown for the canvas fabric.

SSP modes with varying degrees of wavelength confinement (quantified by the plasmon wavenumber βp ranging from 0.25π rad cm⁻¹ to 0.85π rad cm⁻¹) can be achieved by tuning the geometrical parameter h of the structure and using different fabric materials as the intermediate layer as shown in FIG. 12A. To enhance sensitivity, a high degree of wavelength confinement (i.e. a large Bp value) is desired, which can be achieved with larger h.

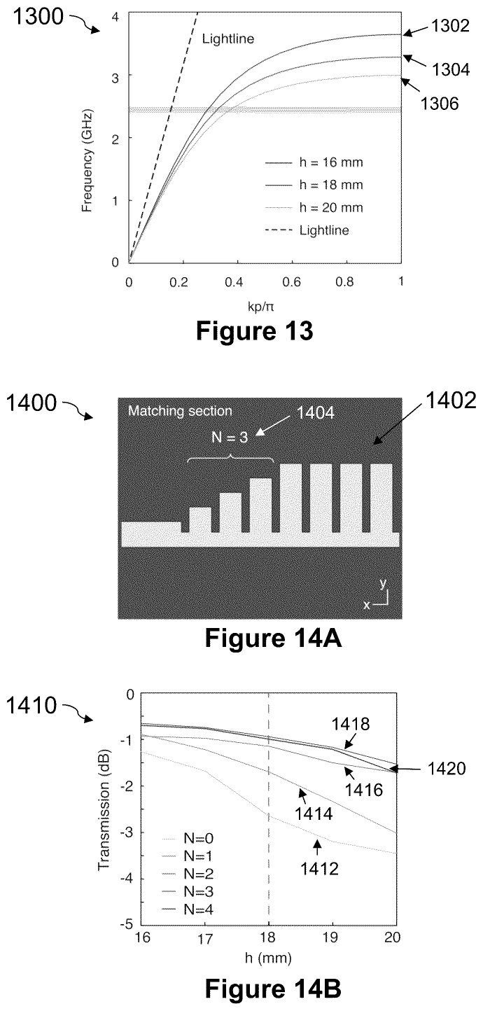

To illustrate this point in relation to the geometrical parameter h, a dispersion graph 1300 for metamaterial textile waveguides with varying geometrical parameter h in accordance with an embodiment is shown in FIG. 13. The dispersion graph 1300 includes a dispersion curve 1302 of a metamaterial textile waveguide having the parameter h=16 mm, a dispersion curve 1304 of a metamaterial textile waveguide having the parameter h=18 mm, and a dispersion curve 1306 of a metamaterial textile waveguide having the parameter h=20 mm. It is clear from the graph 1300 that there is less dispersion for larger h (e.g. comparing the dispersion curve 1302 for h=16 mm with that of the dispersion curve 1306 for h=20 mm).

Referring back to FIG. 12A, a high degree of wavelength confinement (i.e. a large Bp value) can also be achieved by selecting a suitable fabric material for the intermediate non-conductive layer 1108.

There is, however, a design trade-off between wavelength confinement and transmission loss as increasing h results in higher transmission losses. This is shown in relation to FIG. 12B.

FIG. 12B shows the graph 1210 of transmission loss as a function of the textile conductivity at 2.4 GHz for different geometrical parameters h. The transmission loss for h=16 mm is shown in relation to a plot 1212, the transmission loss for h=18 mm is shown in relation to a plot 1214, and the transmission loss for h=20 mm is shown in relation to a plot 1216. As shown in the graph 1210, the larger a value of the geometrical parameter h, the higher is the transmission loss for a given conductivity. As high losses are detrimental to the functionality of the sensor, h=18 cm was selected to maintain a transmission loss of less than 0.3 dB cm⁻¹ when fabricated with a conductive textile (σ=5×10⁵ Sm⁻¹) on a polyester fabric substrate.

To improve a performance of a metamaterial textile waveguide, planar matching sections using gradient corrugated strip with linear taper was designed to realise negligible surface-to-propagation conversion loss from a 50Ω port of the RF frontend 510 of the SDR system 106.

FIGS. 14A and 14B show a diagram 1400 and a graph 1410 to illustrate design for a planar matching section of a metamaterial textile waveguide in accordance with an embodiment.

FIG. 14A shows the diagram 1400 for illustrating a structure of the planar matching section 1402 at an end of the metamaterial waveguide. In the present embodiment, three matching units (i.e. N=3) were used.

FIG. 14B shows the graph 1410 of transmission coefficient as a function of the geometrical parameter h for varying number of matching units, N. The transmission coefficient as a function of h for N=0 is shown in relation to a plot 1412, the transmission coefficient as a function of h for N=1 is shown in relation to a plot 1414, the transmission coefficient as a function of h for N=2 is shown in relation to a plot 1416, the transmission coefficient as a function of h for N=3 is shown in relation to a plot 1418, and the transmission coefficient as a function of h for N=4 is shown in relation to a plot 1420. As shown in FIG. 14B, N=3 provides the highest transmission and it is therefore selected as the number of matching units used.

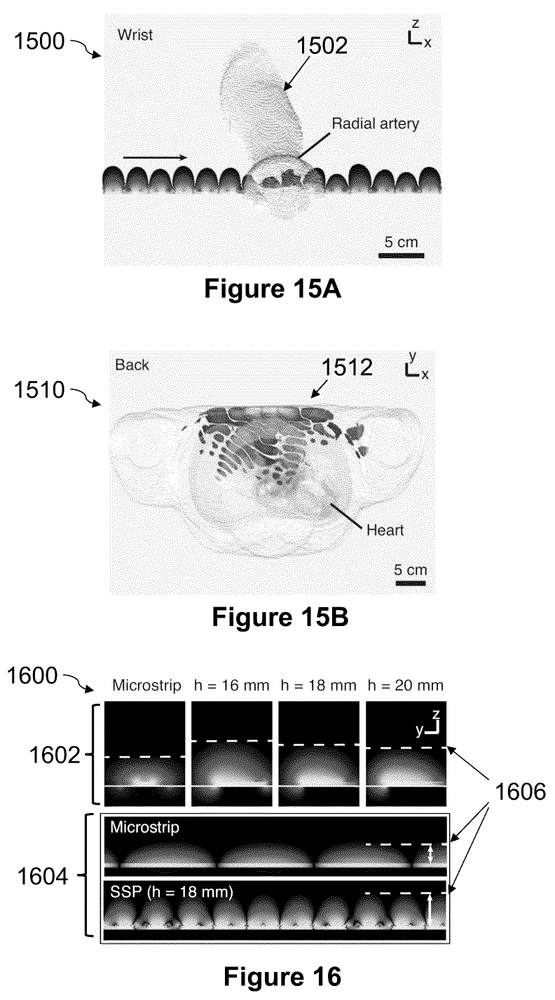

FIGS. 15A and 15B show diagrams 1500, 1510 of full wave simulations during sensing in accordance with embodiments. FIG. 15A shows the diagram 1500 of a full wave simulation of a cross-section of an arm 1502 during radial pulse sensing and FIG. 15B shows the diagram 1510 of a full wave simulation of a cross-section of a human body torso 1512 during heartbeat sensing or respiration sensing.

Computational arm and body voxel models were used to simulate electromagnetic field distributions. The resolution of the voxel was 2 mm×2 mm×2 mm. Cardiac cycle simulations of the radial pulse and heartbeat were conducted by periodic geometrical variations on the voxel models of the radial artery and the heart. The simulated tissue motions lead to changes in effective permittivity, which are demodulated as phase variations Δϕ of the scattering parameter S21.

As shown by the full-wave simulations in relation to FIGS. 15A and 15B, the metamaterial textile sensor of the present embodiments allows confinement of electromagnetic energy on its surface for interactions with the nearby computational arm 1502 and torso 1512 models.

FIG. 16 shows a diagram 1600 of electric field profile in the y-z plane in a top panel 1602 and x-z plane in a bottom panel 1604 of a conventional microstrip line and a metamaterial textile sensor of the present disclosure in accordance with an embodiment. The white dashed lines 1606 show the decay length 3α⁻¹.

The SSP modes can conformally propagate on surfaces of daily objects and interact strongly with the radial artery and the heart through evanescent fields, enabling unobtrusive wrist pulse and heartbeat detection. In contrast, conventional waveguides, such as microstrip lines, lack such an evanescent field, resulting in a lower coupling efficiency with tissues. This is illustrated in relation to FIG. 16 where the decay length 3α⁻¹ is shorter for the microstrip lines than for the metamaterial waveguide. Moreover, the high energy confinement and shortened wavelength of the SSP modes for the metamaterial waveguide significantly enhance the transduction of subtle tissue impedance modulations into larger phase variations of the propagating electromagnetic waves.

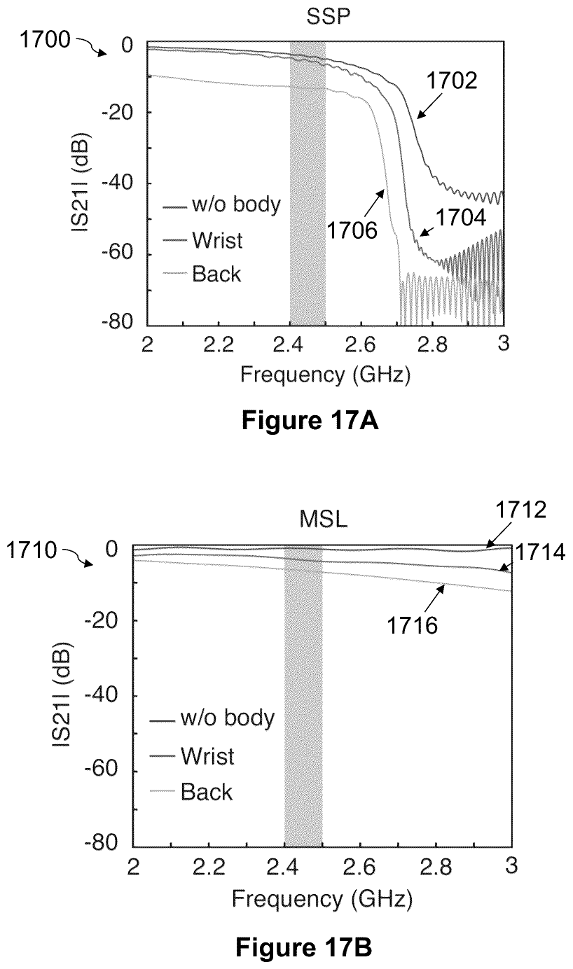

FIGS. 17A and 17B show graphs 1700, 1710 of simulated |S₂₁| transmission spectra without an interaction with a body and due to interactions with different body parts in accordance with embodiments. This also provides evidence for the lower coupling efficiency with tissues between the conventional microstrip lines textile sensor as compared to the SSP metamaterial sensor of the present disclosure.

FIG. 17A shows the graph 1700 of the simulated |S₂₁| transmission spectra for the SSP metamaterial textile sensor comprising the waveguide 1104. The simulated |S₂₁| transmission spectra without an interaction with a body (i.e. free space) is shown in relation to a plot 1702, the simulated |S₂₁| transmission spectra having an interaction with a wrist of the body is shown in relation to a plot 1704, and the simulated |S₂₁| transmission spectra having an interaction with a back of the body is shown in relation to a plot 1706. The simulated |S₂₁| transmission spectra of the graph 1700 was obtained using a U-shaped SSP metamaterial textile sensor, this differs from the results obtained in relation to FIG. 4A which had used a straight SSP metamaterial textile sensor.

FIG. 17B shows the graph 1710 of the simulated |S₂₁| transmission spectra for a microstrip line (MSL) textile sensor. The simulated |S₂₁| transmission spectra without an interaction with a body (i.e. free space) is shown in relation to a plot 1712, the simulated |S₂₁| transmission spectra having an interaction with a wrist of the body is shown in relation to a plot 1714, and the simulated |S₂₁| transmission spectra having an interaction with a back of the body is shown in relation to a plot 1716.

From FIGS. 17A and 17B, it is shown that a coupling efficiency of the SSP metamaterial textile sensor with body tissues is higher than that of the MSL textile sensor, resulting in a greater change in the simulated |S₂₁| transmission spectra in the presence of body tissues for the SSP metamaterial textile sensor as compared to the MSL textile sensor.

To evaluate the enhancement of phase modulations, the responses of the SSP metamaterial textile sensor and that of the microstrip line sensor to pulsating cardiac motions, simulated as geometric variations of the radial artery and the heart in computational voxel models, were compared.

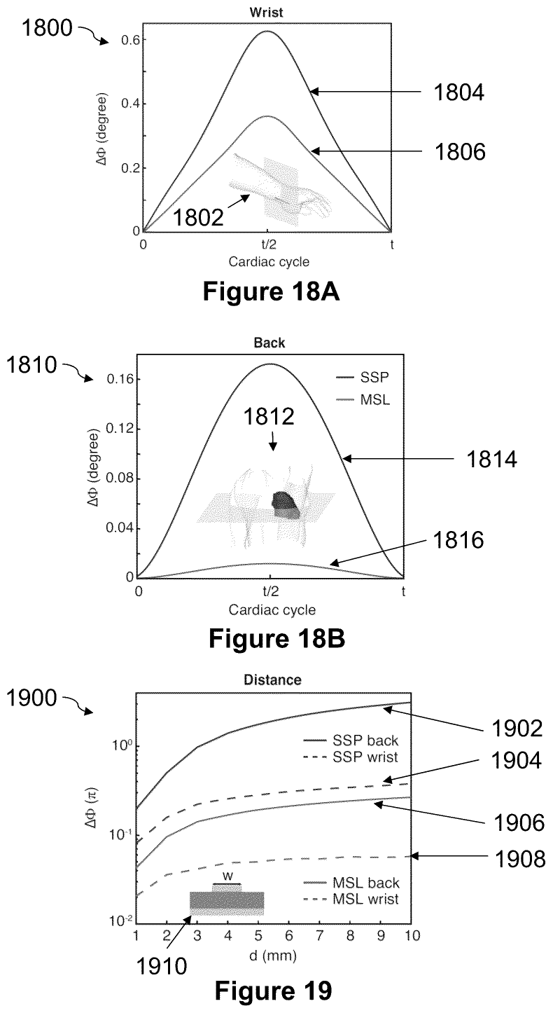

FIGS. 18A and 18B show graphs 1800, 1810 of simulated phase variations Δϕ obtained from the conventional microstrip line textile sensor and the metamaterial textile sensor of the present disclosure in accordance with an embodiment.

FIG. 18A shows the graph 1800 of simulated phase variations Δϕ as a result of a radial pulse within a cardiac cycle. FIG. 18A also shows an inset 1802 of an arm torso simulation model. A plot 1804 shows simulated phase variations Δϕ as a result of a radial pulse within a cardiac cycle for the SSP metamaterial textile sensor while a plot 1806 shows simulated phase variations Δϕ as a result of a radial pulse within a cardiac cycle for the MSL textile sensor.

FIG. 18B shows the graph 1810 of simulated phase variations Δϕ as a result of a heartbeat within a cardiac cycle. FIG. 18B also shows an inset 1812 of a human torso simulation model. A plot 1814 shows simulated phase variations Δϕ as a result of a heartbeat within a cardiac cycle for the SSP metamaterial textile sensor while a plot 1816 shows simulated phase variations Δϕ as a result of a heartbeat within a cardiac cycle for the MSL textile sensor.

As shown in relation to FIGS. 18A and 18B, the simulated phase variations Δϕ due to the radial pulse and the heartbeat for the SSP metamaterial textile sensor were observed to be enhanced by 2 and 15 times, respectively, as compared to that for the conventional microstrip line textile sensor. The sensitivity can be tuned by varying the amount of electromagnetic energy coupled into the body and is controlled by the distance of the body from the SSP metamaterial textile sensor. This is shown in relation to FIG. 19.

FIG. 19 shows a graph 1900 of simulated phase variations Δϕ as a function of separation distance d at the back and the wrist of a body in accordance with an embodiment, where the distance d is varied from 1 mm to 10 mm. The simulated phase variations Δϕ as a function of separation distance d are shown for both the conventional microstrip line (MSL) textile sensor and the SSP metamaterial textile sensor of the present disclosure. This is shown in FIG. 19, where a plot 1902 shows simulated phase variations Δϕ as a function of separation distance d at the back of the body for the SSP metamaterial textile sensor, a plot 1904 shows simulated phase variations Δϕ as a function of separation distance d at the wrist of the body for the SSP metamaterial textile sensor, a plot 1906 shows simulated phase variations Δϕ as a function of separation distance d at the back of the body for the MSL textile sensor and a plot 1908 shows simulated phase variations Δϕ as a function of separation distance d at the wrist of the body for the MSL textile sensor. An inset 1910 shows a cross-section of the MSL textile sensor with a width of the MSL, w=6.8 mm. This parameter was used for the simulations in relation to the MSL textile sensor. FIG. 19 shows that the SSP metamaterial textile sensor exhibits 10 times larger phase variations as compared to the MSL textile sensor, indicating higher achievable sensitivity and broader tunability for the SSP metamaterial textile sensor.

Due to the near-field nature of the present sensing approach utilising the SSP metamaterial textile sensor, localized multi-point sensing of vital signs can be achieved through strategic design and placement of the sensor on ordinary daily objects.

The SSP metamaterial textile sensor is robust in folding and bending, therefore bending of the SSP metamaterial textile sensor in two different planes were investigated.

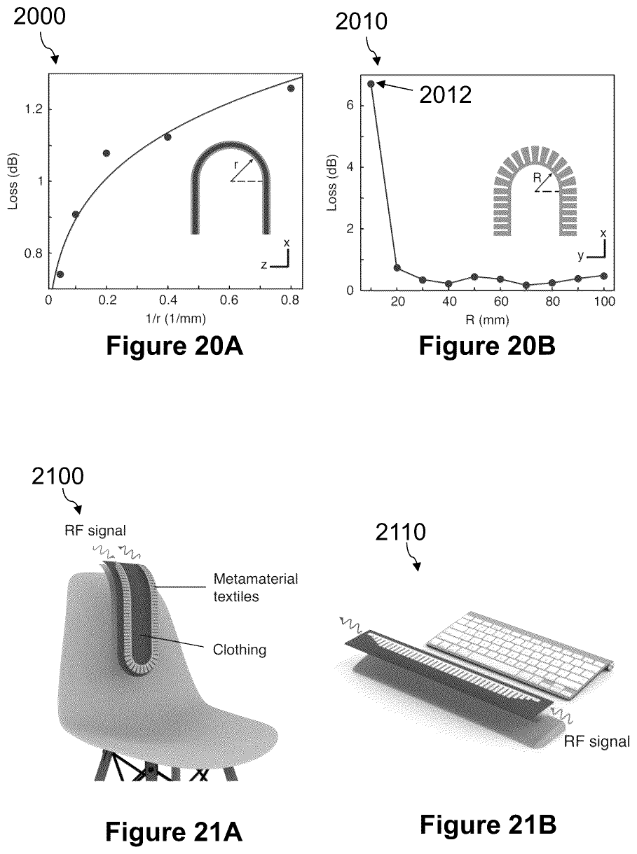

FIGS. 20A and 20B show plots 2000, 2010 of transmission loss of the SSP metamaterial textile sensor as a function of a bending radius in accordance with embodiments for the x-z plane and the x-y plane, respectively. As shown in FIGS. 20A and 20B, the transmission loss of the SSP metamaterial textile sensor decreases as the bending radius increases. The plots 2000, 2010 show that the transmission loss of the SSP metamaterial textile sensor is kept low for a wide range of bending radius in both the x-z and y-z planes, respectively (except when the bending is too extreme, for example as shown by the first data point 2012 of FIG. 20B). The SSP metamaterial textile waveguide is therefore robust to bending, and this makes it a suitable candidate for conformally integrating into many different types of surfaces/accessories for non-contact sensing without any significant degradation in performance.

In the present embodiment for multi-point sensing, two sensor designs—a straight one and a U-shape one—were used to facilitate conformal and versatile incorporation into tables and chairs of diverse shapes and sizes. The two sensor designs are shown in relation to FIGS. 21A and 21B. FIGS. 21A and 21B show diagrams 2100, 2110 of two SSP metamaterial textile sensor designs in accordance with embodiments, where FIG. 21A shows the U-shape sensor design for use on a chair and FIG. 21B shows the straight sensor design for use on a table.

FIG. 22 shows an illustration 2200 of a vital sign data collection setup using the system 100 of FIG. 1A in accordance with an embodiment. In particular, respiration and heart signals were measured from the back of the body 2202 via channel 1 (CH1) 2204 using the U-shape sensor provided on a backrest of an office chair, and radial pulse signal from the wrist of the body 2202 was measured via channel 2 (CH2) 2206 using the straight sensor provided on the desk. The Doppler phase variations induced by the respective physiological actions were demodulated at the SDR 2208 and bandpass filtered in the computer to separate the respiration signals (0.1-0.8 Hz) and the heart signals (0.9-5.0 Hz) in CH1 2204, as well as the radial pulse signals (0.9-5.0 Hz) in CH2 2206. Beat detection algorithms 2210 were developed for respiration, heartbeat and pulse signals to extract the respective beat-to-beat rates. This is described in relation to FIG. 6, and is shown in FIG. 22 where the respiration rate 2212 and the heart rate 2214 can be extracted from the signals received from CH1 2204, while the radial pulse rate 2216 can be extracted from the signal received from CH2 2206. Heartbeat and radial pulse data collected are aligned by the machine learning model, LSTM aligner 2218, for calculating pulse transit time (PTT) which is used for calculating blood pressure (BP) values.

To collect the respiration and heartbeat data, the U-shape SSP metamaterial textile sensor was attached to the backrest of the office chair. The participants were instructed to sit upright on the chair and rest lightly on the backrest. The sensor was connected to CH1 2204 of the SDR system 2208, and the Doppler phase signals were recorded using the computer. For the collection of data, the participants were asked to breathe normally while minimizing limb movements. For each of the respiration and heartbeat sensing evaluations, the reference signals were respectively collected using a respiratory effort transducer (SS5LB, BIOPAC Systems, Inc) fastened around the participant's thorax, or a 3-lead ECG transducer (SS2LB, BIOPAC Systems, Inc.) attached to the participant's legs and right arm. The synchronization of recorded physiological signals from the sensor measurements and the reference measurements was performed using system timestamps obtained from screen video recordings.

For radial pulse data collection, the straight SSP metamaterial textile sensor was placed on the office table and connected to CH2 2206 of the SDR system 2208. The participants were instructed to place either their right or left wrist on the straight SSP metamaterial textile sensor, with the radial artery directly on top of the conductive textile, and were asked to breathe normally while minimising movements. The Doppler signals from the radial arterial pulse motions were recorded in the same manner as the respiration or heartbeat evaluation experiment. The reference pulse signals were collected simultaneously using a finger PPG sensor (SS4LA, BIOPAC Systems, Inc.) fastened on the index finger on the same side as the wrist under measurement.

FIG. 23 shows plots 2300 of sample data collected from the two sensor channels, CH1 2204 and CH2 2206, using the vital sign data collection setup of FIG. 22 in accordance with an embodiment.

The raw data 2302 from CH1 shows a superposition of respiration and heartbeat information, whereas the raw data 2304 from CH2 shows clear radial pulse beats. The raw data 2302, 2304 (i.e. the time-varying phase shift signals ϕ_{D} (t)) are processed by bandpass filtering to obtain a respiration signal 2306 (also known as a time-varying respiration phase signal in the present disclosure), a heart signal 2308 (also known as a time-varying heart phase signal in the present disclosure), and a pulse signal 2310 (also known as a time-varying radial pulse phase signal in the present disclosure). Beat-to-beat respiration, heartbeat and pulse rates can be obtained using the beat detection algorithms as afore-described.

FIG. 23 shows qualitatively the detected breaths 2312, heartbeats 2316 and pulses 2320 on recorded signal segments, overlaid with true reference breaths 2314, reference ECG R-peaks 2318 and reference PPG peaks 2322 obtained from their respective references, namely respiratory belt, electrocardiography (ECG) and finger photoplethysmography (PPG). For the respiration signal 2306, it can be observed that the respiratory peaks in the measured signal closely follow the reference signal, suggesting good temporal agreement. For the heart signal 2308 and the pulse signal 2310, however, a consistent temporal shift between the detected and the reference beats can be observed due to the different choices of beat markers in each waveform. In particular, while the ECG R-wave peaks 2318 and PPG peaks 2322 are conventionally used as beat locations, the beat-to-beat algorithms of the present disclosure detect the positive zero-intercepts as beats.

To validate the accuracy of the SSP metamaterial textile sensors, three independent experiments with human volunteers were performed to benchmark the respiration rate, heart and pulse beat-to-beat intervals (BBIs) against their respective reference measurements. The degree of agreement between the sensor measured beats and the reference beats was evaluated by performing Bland-Altman analyses on all recorded BBIs for respiration, heartbeat and radial pulse as shown in relation to FIGS. 24A to 24C.

For each experiment, healthy participants (20-35 years of age) with no known pre-existing cardiopulmonary conditions were recruited. The number of participants for each evaluation experiment is provided in Table 1 below:

TABLE 1
Number of participants for each evaluation experiment

| Validating | | | |
|---|---|---|---|
| Experiment | Male | Female | Total participants |
| Respiration | 3 | 2 | 5 |
| Heartbeat | 5 | 5 | 10 |
| Arterial pulse | 5 | 5 | 10 |

Participants wore normal office attire, such as T-shirts and blouses. All experiments were performed in an open dry-lab space, in the presence of other lab members unrelated to the experiments. Each trial included the recording of 10 minutes of data per participant.

Reference data were measured using the BIOPAC MP46 2-channel data acquisition (DAQ) unit connected to the relevant compatible respiration, ECG or PPG transducers, powered by a computer and controlled with the BIOPAC Student Lab Software.

FIGS. 24A, 24B and 24C show Bland-Altman plots 2400, 2410, 2420 of beat-to-beat intervals obtained from reference measurements and from using the metamaterial textile sensor of the present disclosure in accordance with an embodiment. FIG. 24A shows the Bland-Altman plot 2400 for a respiration rate. FIG. 24B shows the Bland-Altman plot 2410 for a heart rate, and FIG. 24C shows the Bland-Altman plot 2420 for a radial pulse rate. As described previously, the Bland-Altman plot 2410 of FIG. 24B uses data from 10 subjects including the data for the Bland-Altman plot 820 in relation to FIG. 8C.

Each of the Bland-Altman plots 2400, 2410, 2420 show the bias (or mean) 2402, 2412, 2422 and the limits of agreement (LoA) 2404, 2414, 2424 for the respiration rate, heart rate and radial pulse rate, respectively. The insets 2406, 2416, 2426 show the number of male and female participants for the plots 2400, 2410, 2420, respectively.

As shown in the Bland-Altman plots 2400, 2410, 2420 of FIGS. 24A, 24B and 24C, good agreements for all sensing targets with low biases of 0.627 ms, −0.143 ms and 0.128 ms, were observed for the respiration rate, the heart rate and the radial pulse rate, respectively. Narrow limits of agreement (LoA) denoted by the dashed lines 2404, 2414, 2424 were also observed.

Along with the respiration rate and heart rate, blood pressure (BP) is another vital parameter of a person's long-term well-being and is a particularly insightful indicator of cardiovascular health that is routinely monitored by healthcare providers. In current BP measurement practices, cuff-based methods such as auscultation or oscillometry are the most popular, particularly with oscillometry being the “clinical standard” as it is non-invasive and well-validated. Nevertheless, the use of cuffs in BP measurement is inconvenient, time-consuming and non-continuous, leading to unsatisfactory detection and management of hypertension and related cardiovascular diseases. As a result, there is a strong need for ubiquitous, continuous BP monitoring techniques that are convenient, unobtrusive and low cost, which can be used during daily activities with minimal disruption.

In the present disclosure, as shown in relation to the illustration 2200 of FIG. 22, the system 100 is adapted to obtain highly accurate vital signs from multiple locations on the body and this can be used to provide ubiquitous, cuffless and continuous BP monitoring during daily living. This is described in relation to FIGS. 25 to 36 below.

Particularly, the present system 100 uses pulse transit time (PTT), which is defined as the time taken for a cardiac pulse wave to travel between two arterial sites on the body, for measuring BP without cuffs. Conventional PTT measurements mostly rely on ECG and PPG sensors which require mechanically stable contact with the skin. Further, other contactless vital sign monitoring techniques, such as those using radiative RF waves, do not possess the necessary spatial selectivity for PTT measurements, and thus are unsuitable for BP measurements. In contrast, the present system 100 includes SSP metamaterial textile sensors which are adapted for non-radiating sensing over a localised area/space. This provides the avenue for accurate cuffless BP monitoring.

Utilizing this multi-point sensing capability of SSP metamaterial textile sensors, the sensor placement as illustrated in relation to FIG. 22 allows for simultaneous measurement of heart and pulse signals that can provide additional temporal information related to each heartbeat. Conventionally, PTT is measured as the time delay between the ECG R-peak and a particular feature of the PPG waveform, such as the foot or the peak. As measured heart signal and radial pulse signal are related to their respective reference (i.e. ECG and PPF, respectively) on a physiological level (i.e. originating from the same physiological processes), the timing of the ECG R-peaks and PPG maximum first derivative (MFD) points used for obtaining the PTT can thus be inferred from the measured heart signal and the measured radial pulse signal, respectively. To perform PTT measurement adhering to conventional standards, a machine learning model had been developed to align the prominent features (e.g. peaks) of the measured heart and radial pulse signals with their respective reference features.

For the machine learning model, the signal alignment was formulated as a sequence-to-sequence learning task to convert measured Doppler phase signals (or time-varying phase shift signals), which represent either heart or radial pulse information depending on the originating sensor channel (i.e. CH1 or CH2), into a related time series with temporal features closely matching their respective references. The algorithm includes two long short-term memory (LSTM) networks followed by fully connected (FC) layers working independently on each sensor channel, as shown in FIG. 25.

FIG. 25 shows a diagram 2500 illustrating a machine learning model comprising long short-term memory (LSTM) networks and fully-connected (FC) layers for performing heart signal (CH1) and pulse signal (CH2) alignment to obtain pulse transit time (PTT) for blood pressure measurements in accordance with an embodiment.

To begin, measured Doppler phase signals (i.e. the time-varying phase shift signals ϕ_{D} (t)) were received from the sensor channels. These sensor outputs (i.e. the time-varying phase shift signals ϕ_{D} (t)) from CH1 and CH2 were bandpass filtered from 0.9 to 5 Hz to obtain input heart signal 2502 for CH1 and input pulse signal 2504 for CH2 as shown in FIG. 25. The reference ECG signal 2506 and the reference PPG signal 2508 are also shown as reference. Prior to processing by the machine learning model, as shown in FIG. 25, peaks of the input heart signal 2502 are not aligned with the ECG R-peaks of the reference ECG signal 2506.

The input heart signal 2502 is then left-shifted by 0.2-second (i.e. time-advance) with respect to the reference ECG signal 2506 in a step 2510 to form a processed time-varying heart phase signal, while a time-derivative of the first order is taken for the input pulse signal 2504 in a step 2512 to form a processed time-varying radial pulse phase signal. These processed time-varying heart phase signal and the processed time-varying radial pulse phase signal are then input into their respective sequence input layers 2514 for transforming them to sequences of a same length, prior to providing their sequenced outputs to their respective long short-term memory (LSTM) networks 2516 and subsequently to their respective fully-connected (FC) layers 2518 as shown in FIG. 25. The processing layers (i.e. related to the processing step 2510 for left-shifting the input heart signal 2502 and the processing step 2512 for apply the time-derivative on the input pulse signal 2504), the sequence input layers 2514, the LSTM networks 2516 and the FC layers 2518 are collectively known as the “LSTM aligner” in the present disclosure and forms the machine learning model. The LSTM aligner outputs aligned time-varying heart phase signal 2520 and aligned time-varying radial pulse phase signal 2522 with their peaks aligned to the reference ECG R-peaks of the reference ECG signal 2504 and the PPG MFD points of the time-derivative of the reference PPG signal 2524, respectively as shown in FIG. 25. Beat-to-beat PTT 2526 can then be calculated as a time delay between the detected peaks of these aligned time-varying heart phase signal 2520 and aligned time-varying radial pulse phase signal 2522 as shown in FIG. 25.

The computer 108 can be configured to perform PTT calculation on the aligned time-varying heart phase signal 2520 and aligned time-varying radial pulse phase signal 2522. In the present embodiment, for each located peak of the aligned time-varying heart phase signal 2520, the computer 108 stores an algorithm which is adapted to search, within its ensuing time window of 0.15-0.4 s, a corresponding local maximum of the corresponding aligned time-varying radial pulse phase signal 2522. The algorithm may also be configured to search for and remove falsely detected peaks by detecting outliers in the BBIs in an intermediate step. For example, the outliers can be defined as values that are more than five scaled median absolute deviations from the median of all BBIs in an epoch or recording, although other definitions for the outliers can be used in other embodiments.

FIG. 26 shows a Bland-Altman plot 2600 of the estimated PTT from testing data using the machine learning model of FIG. 25 in accordance with an embodiment. The Bland-Altman plot 2600 of the estimated PTT was formed using the LSTM aligner outputs and the reference ECG and PPG measurements obtained from testing data which was collected from 2 subjects for 10 minutes each.

The Bland-Altman plot 2600 in FIG. 26 shows a small mean bias 2602 compared to the reference at −0.192 ms with the data points lying within the limits of agreement (LoA) 2604 of about 50 ms.

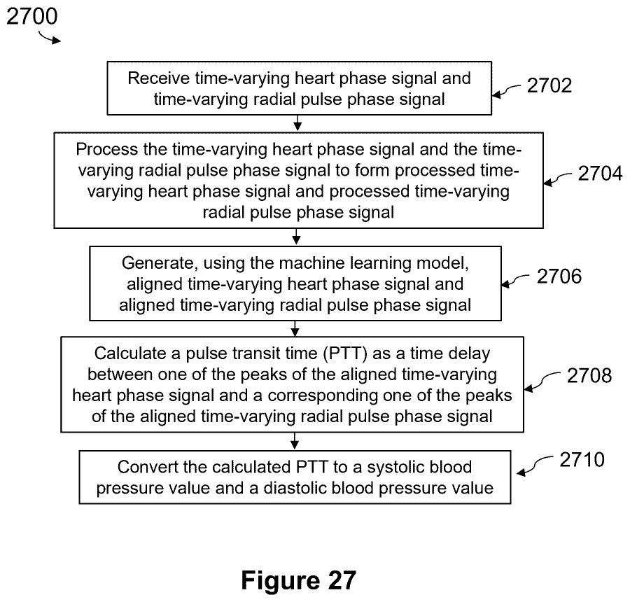

FIG. 27 is a flowchart showing steps of a computer-implemented method 2700 for generating aligned time-varying heart phase signal and aligned time-varying radial pulse phase signal for calculating a pulse transit time (PTT) for converting to systolic and diastolic blood pressure values in accordance with an embodiment. This provides a summary to the aforementioned process as described in relation to FIG. 25.

In a step 2702, time-varying heart phase signal and time-varying radial pulse phase signal are received. This corresponds to the input heart signal 2502 and input pulse signal 2504 which were obtained by bandpass filtering their respective time-varying phase shift signal from 0.9 Hz to 5 Hz.

In a step 2704, the time-varying heart phase signal and the time-varying radial pulse phase signal are processed to form processed time-varying heart phase signal and processed time-varying radial pulse phase signal, respectively. This may include left-shifting the time-varying heart phase signal by a predetermined amount of time and differentiating the time-varying radial pulse phase signal with respect to time to generate a time derivative of the time-varying radial pulse phase signal.

In a step 2706, aligned time-varying heart phase signal 2520 and aligned time-varying radial pulse phase signal 2522 are generated for each of the time-varying heart phase signal and the time-varying radial pulse phase signal using the machine learning model which comprises a long short-term memory (LSTM) network followed by a fully connected (FC) layer. Particularly, peaks of the aligned time-varying heart phase signal 2520 correspond to the electrocardiography (ECG) R-wave peaks and peaks of the aligned time-varying radial pulse phase signal 2522 correspond to the photoplethysmography (PPG) maximum first derivative (MFD) points, which can be used for subsequent PTT calculation.

In a step 2708, the pulse transit time (PTT) is calculated as a time delay between one of the peaks of the aligned time-varying heart phase signal 2520 and a corresponding one of the peaks of the aligned time-varying radial pulse phase signal 2522. In an embodiment, for each located peak of the aligned time-varying heart phase signal 2520, the computer 108 stores an algorithm which is adapted to search, within its ensuing time window of 0.15-0.4 s, a corresponding local maximum of the corresponding aligned time-varying radial pulse phase signal 2522.

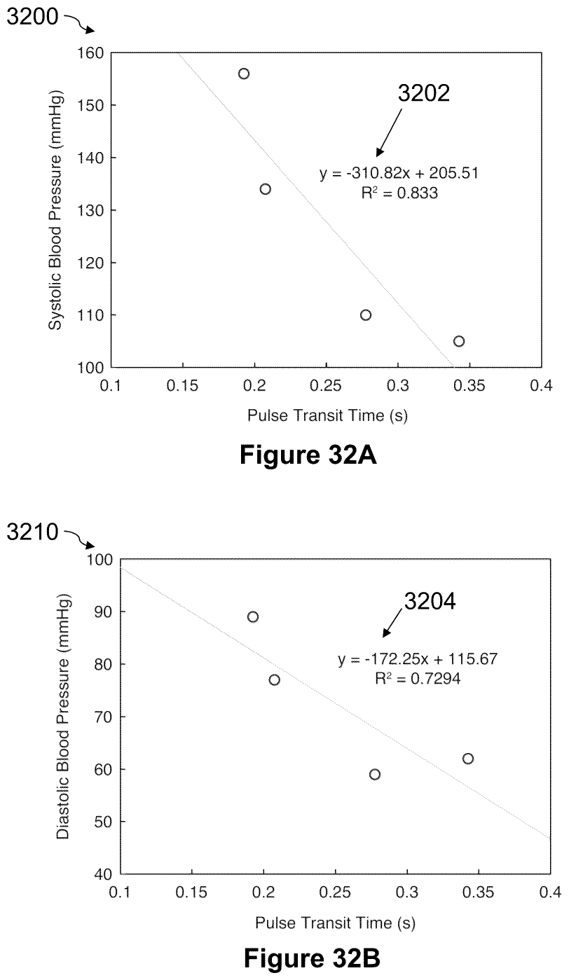

In a step 2710, the calculated PTT from the step 2708 is converted to a systolic blood pressure value and a diastolic blood pressure value. This may involve using a linear PTT-blood pressure relationship. This is further discussed in relation to FIGS. 32A and 32B below.

In order for the machine learning model to provide acceptable outputs in relation to aligning the input heart signal 2502 and input pulse signal 2504 for PTT calculation, the machine learning model has to be trained to form a trained machine learning model.

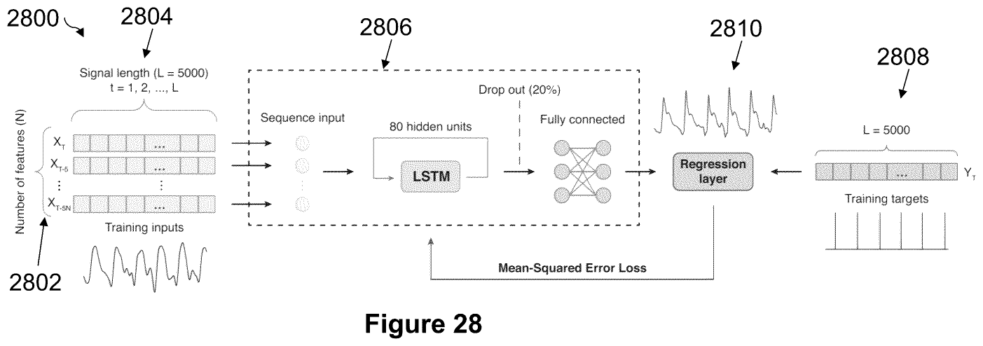

FIG. 28 shows a diagram 2800 illustrating an architecture and training of one of the LSTM aligner channels of the machine learning model of FIG. 25 in accordance with an embodiment. Although the machine model as shown in FIG. 25 performs heart signal (CH1) and pulse signal (CH2) alignment through two separate LSTM-based networks, only one LSTM-based network is shown here for succinctness.

Physiological data (e.g. training heart data and training pulse data) were collected during accuracy evaluation experiments from all subjects and these were used as training data for the LSTM aligner. Two separate datasets to train the respective networks for heartbeat and pulse alignment were constructed. Training time-varying heart signals and training time-varying radial pulse signals were obtained by bandpass filtering their corresponding training time-varying phase signals from 0.9 to 5 Hz. The input features 2802 were constructed by first generating incrementally left-shifted copies of the training time-varying heart signals and training time-varying radial pulse signals in steps of 5 samples until the desired number of features is obtained. In the present embodiment, the number of features N is 5 and 15 for heart signal alignment (CH1) and pulse signal alignment (CH2), respectively.

Each of the features was split into a 5000-sample non-overlapping window 2804, corresponding to 12.5 seconds. This produced a total training sample size of N×5000 which is the size of one training sample. Multiple training samples this size were obtained from each Subject ID as illustrated in Table 3 below. The heart signal features were additionally subjected to a 0.2 s left shift before forming the 5000-sample non-overlapping window 2804. For the pulse signal features, the time-derivative of the pulse signal features were taken prior to forming the 5000-sample non-overlapping window. The testing dataset for evaluating the LSTM aligner was constructed separately using additional dual-channel data collected from 2 healthy volunteers for a total of 10 minutes. All data processing, network training and evaluation were performed in MATLAB (R2021a, The MathWorks, Inc.).

The training input features are provided to a LSTM-based network 2806 (comprising a sequence input layer, a LSTM layer and a fully-connected (FC) layer) and output signals produced by the LSTM-based network 2806 are transformed sequences of the same length. In the LSTM-based network 2806, the sequence layer is used to feed the training samples into the LSTM layer (80 hidden units), followed by a 20% dropout and the FC layer. The training targets 2808 are the corresponding 5000-sample windows ECG R-peak mask or the time-derivative of the PPG. The LSTM-based network 2806 is trained or optimized by minimising the mean-square error (MSE) loss between the output signals and the training targets using a regression layer 2810. Examples of these signals are shown in FIG. 28 in relation to their respective network blocks. Each of the LSTM-based networks for the heart signal (CH1) and the pulse signal (CH2) alignment were trained separately using their respective training and target data sets.

The LSTM aligner's ability to generalize across unseen subjects was evaluated using a subject-based cross-validation process. For every subject, each LSTM-based network is trained using the data from the other 9 subjects and then evaluated on that subject, thus forming a 10-fold cross-validation procedure. Lastly, the respective networks are trained on all training data, followed by evaluation on the testing dataset.

The LSTM-based network parameters are shown in Table 2 for each of the LSTM-based network for CH1 and CH2.

TABLE 2
LSTM-based network parameters

| | | | Feature | |
|---|---|---|---|---|
| | | | number | Hidden |
| Network | Training Input | Training target | (N) | units |
| CH1 | SSP | ECG R-peak mask | 5 | 80 |
| CH2 | Derivative SSP | Derivative PPG | 15 | 80 |

Table 3 showing the number of training samples by Subject ID is provided below:

TABLE 3
Training samples by Subject ID

| Subject ID | 1 | 2 | 3 | 4 | 5 | 6 | 7 | 8 | 9 | 10 | Total |
|---|---|---|---|---|---|---|---|---|---|---|---|
| Pulse | 45 | 45 | 45 | 45 | 45 | 45 | 45 | 45 | 41 | 40 | 441 |
| Heart | 45 | 45 | 45 | 45 | 40 | 40 | 40 | 40 | 40 | 40 | 420 |

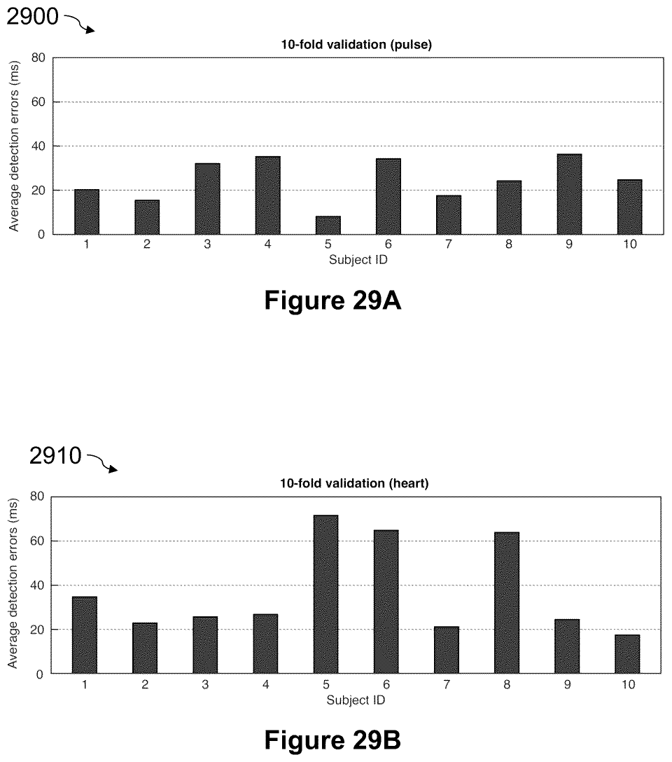

FIGS. 29A and 29B show plots 2900, 2910 of sample data for validating the machine learning model of FIG. 25 for pulse signal alignment and heart signal alignment in accordance with an embodiment.

FIG. 29A shows the plot 2900 of average detection errors against Subject ID for pulse signal alignment and FIG. 29B shows the plot 2900 of average detection errors against Subject ID for heart signal alignment. As shown in FIGS. 29A and 29B, the average detection errors are below 40 ms across all Subject IDs for the pulse signal alignment, and the average detections errors are below 80 ms across all Subject IDs for the heart signal alignment.

With the sensor setup as shown in FIG. 22 and utilising the machine learning model as described in relation to FIG. 25, seamless integration of the SSP sensors in ordinary physical spaces can be achieved and an ambient sensing approach can be realised for unobtrusive, ubiquitous and convenient health monitoring without requiring any active participation or effort from users. Consequently, ambient sensors have become increasingly relevant for healthcare, as they allow for the collection of continuous health data over the long term to provide a more comprehensive understanding of an individual's health trends and patterns, making them highly suitable for preventative health monitoring and early disease detection.

This is particularly important in the modern workplace environment where sedentary lifestyles have become pervasive among adults, as shown by a large number of hours spent sitting at a desk. With higher amounts of sitting time associated with elevated risks of cardiovascular diseases and mortality, long-term physiological monitoring in the office may serve as an effective management strategy to improve the overall public health standards.

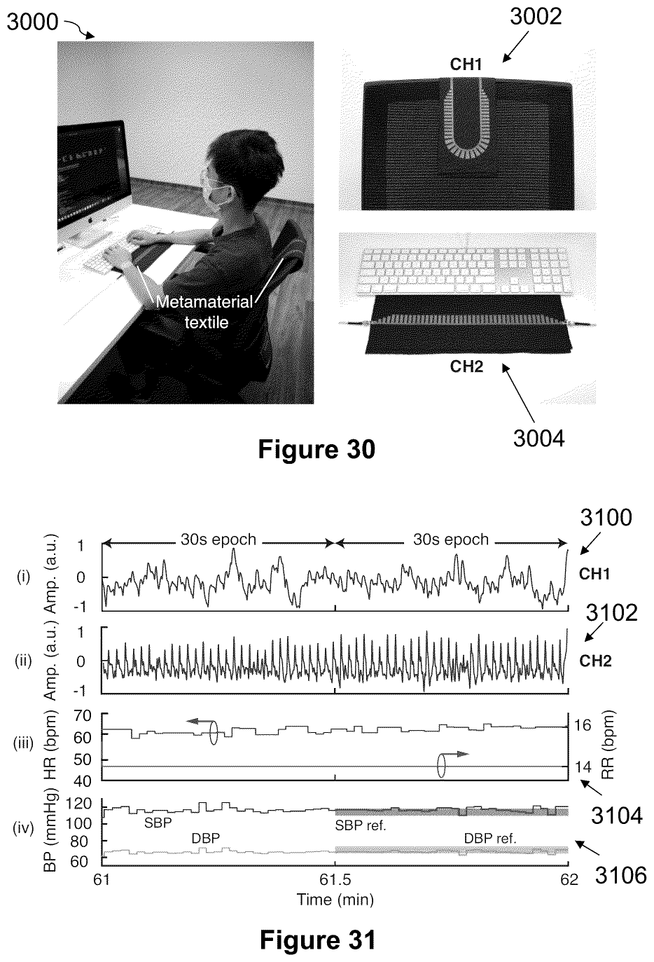

FIG. 30 shows photographs 3000, 3002, 3004 of a multiplexed health monitoring in an office environment using a dual-channel metamaterial textile sensor system in accordance with an embodiment. As shown in the photographs 3002 and 3004, a U-shaped metamaterial textile sensor connected to CH1 is provided on a chair backrest, while a straight metamaterial textile sensor connected to CH2 is provided on a desk in front of a keyboard for recording data simultaneously and continuously for 100 min. The experimental setup as exemplified in relation to FIG. 30 was used to perform ambient contactless sensing of multiple health metrics on a healthy male volunteer over a typical 100-minute working session.

By definition, ambient sensors are ubiquitous and responsive to users while not imposing restrictions on their movements or behaviour. The experiment performed therefore adhered to the intended use case by simulating real-world working conditions, where the human subject and other workers could move about freely in an office space. Particularly, the subject was instructed to sit on the chair and perform regular tasks on the computer such as reading, using the mouse and typing on the keyboard as required during the normal course of work. Movements of the arms and legs and other activities such as drinking, talking with others as well as getting up from the sitting positions were not restricted. During this period, other workers within the same office space were also free to move about. Additionally, the subject's body and wrist positions were not restricted to a particular location for continuous measurement. Instead, the dual-channel metamaterial textile sensor system was implemented to perform its sensing functions whenever the right conditions were met for the respective channels to detect the presence of the subject's vital signs.

Data was collected continuously from both channels throughout the entire duration of the experiment. A digital sphygmomanometer (OMRON HEM-7156-A) was used to provide reference BP measurements from the left arm in approximately 20-minute intervals, for a total of 5 measurements. At 2 minutes prior to each reference measurement, the subject was instructed to place his right wrist on CH2 for pulse sensing until the reference BP values have been successfully recorded. To calculate BP values from PTTs, a linear relationship between PTT and BP obtained from a separate calibration by least squares regression of measured PTT and BP pairs was used in the present embodiment.

FIGS. 32A and 32B show graphs 3200, 3210 of linear regression for estimating blood pressure using calculated PTT values in accordance with an embodiment. FIG. 32A shows the graph 3200 of linear regression for estimating systolic blood pressure and FIG. 32B shows the graph 3210 of linear regression for estimating diastolic blood pressure.

For each data point, the systolic and diastolic BP values were measured by a commercial digital BP monitor (OMRON) and the PTT value was calculated from using sensor outputs as described in relation to FIG. 25. BP of the subject was varied by means of moderate exercise followed by a period of rest. The equations 3202, 3204 of the best-fit lines by linear regression for the systolic blood pressure and the diastolic blood pressure respectively were used in subsequent BP-estimation from calculated PTT values.

Referring back to multiplexed health monitoring in an office environment, as the subject was allowed to move freely and random movements of the subject may cause spikes in the sensor outputs that mask the desired signals, epochs with large movement present were excluded from the rate calculations. In the present embodiment, an algorithm was implemented to automatically detect “absent” epochs, in which the subject was not seated on the chair, and “continuous BP” epochs, in which high-quality data was available from both sensor channels for BP calculation.

In the present embodiment, absence detection and continuous BP period detection were performed in 30 s epochs. For absence detection, a threshold was implemented on the spectral power stored in the 99% occupied bandwidth (OBW) of the filtered, band-passed sensor output from CH1. In the present embodiment, the threshold was chosen as −45 dB, and below which the epoch is labelled as “absent”. It should be appreciated that a different threshold may be chosen in other embodiments.

The auto-detection of “continuous BP” epochs is based on the fact that if the heart and pulse signals are simultaneously present in an epoch, then the heart rate (HR) and pulse rate (PR) calculated from the respective signals should be equal. In the present embodiment, to implement the auto-detection of “continuous BP” epochs, the absence detection method as described above was first used to identify whether signals are present in both channels. The mean HR and PR were then calculated for the identified epochs (i.e. signals are present in both channels) and their absolute difference was taken as a similarity metric. An epoch is selected as a candidate epoch for continuous BP detection if the absolute difference is less than 2 beats per minute (bpm). To further improve robustness, a signal quality metric was defined for both signals in each candidate epoch, based on the rationale that in a high-quality signal, the mean HR or PR calculated by way of the BBIs should be consistent with that calculated as the number of detected beats per minute, as:

$${Q}_{e} = \frac{\frac{N}{{t}_{e}}}{\frac{1}{N - 1} {\sum }_{t = 1}^{N - 1} \frac{60}{I ( i )}}$$

where N is the number of detected beats in an epoch, tₑ the length of the epoch in minute, and I is the BBI calculated from each successive pair of detected beats in seconds. Finally, the candidate epoch is selected (the selected candidate epoch being known as a detection epoch in the present disclosure) for continuous BP detection if Qₑ>0.5 for both heart and pulse signals in the candidate epoch.

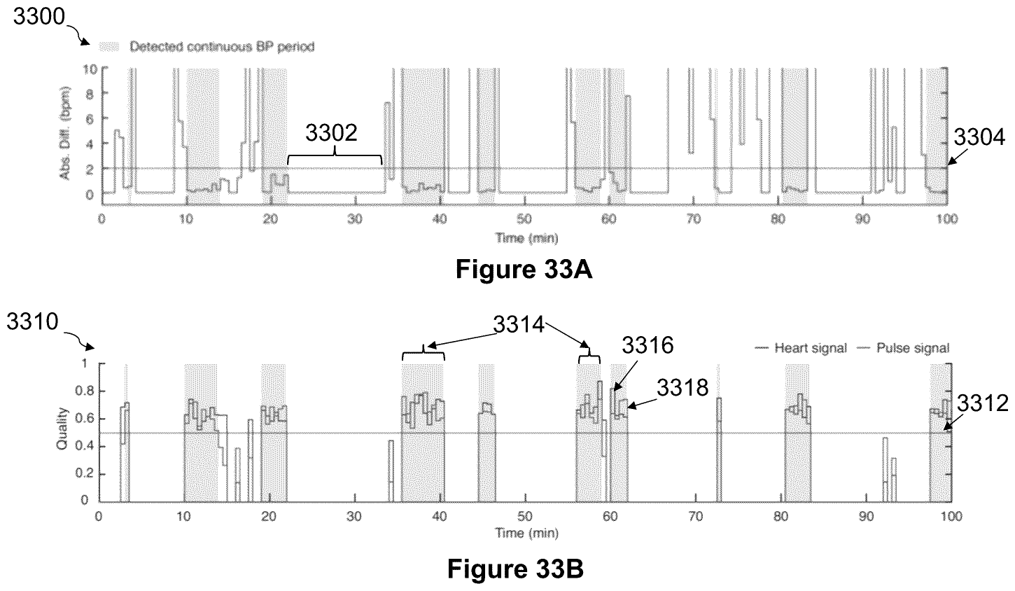

FIGS. 33A and 33B show graphs 3300, 3310 in relation to selecting candidate epoch for continuous blood pressure detection in accordance with an embodiment.

FIG. 33A shows the graph 3300 for identifying candidate epochs for blood pressure sensing using an absolute difference between a mean heart rate (HR) and a mean pulse rate (PR) for each epoch. The graph 3300 includes the absolute difference for the y-axis and time for the x-axis. The graph 3300 shows the data collected over a period of 100 minutes. The epochs 3302 identified as “absent” for either heart or pulse signal are assigned a 0 value. The threshold was set to 2 bpm as indicated by a line 3304. Epochs in which the absolute difference is below this threshold is considered a “continuous BP” candidate (or a candidate epoch).

FIG. 33B shows the graph 3310 for selecting candidate epochs identified in FIG. 33A for continuous blood pressure detection using a signal quality metric (Qₑ). The signal quality metric was calculated for all “continuous BP” candidate epochs identified in the graph 3300. Non-candidate epochs are assigned a Qₑ value of 0. The threshold is set to be 0.5 as indicated by the line 3312. Epochs with both heart signal 3316 and pulse signal 3318 quality above this threshold were identified as “continuous BP” epochs (or detection epochs) as indicated by the shaded boxes 3314. PTT and BP were then calculated for these “continuous BP” epochs.

FIG. 31 shows graphs 3100, 3102, 3104, 3106 of normalized outputs from both channels CH1 and CH2, and the detected vital signs including heart rate (HR), respiratory rate (RR), systolic blood pressure (SBP) and diastolic blood pressure (DBP) in accordance with an embodiment.

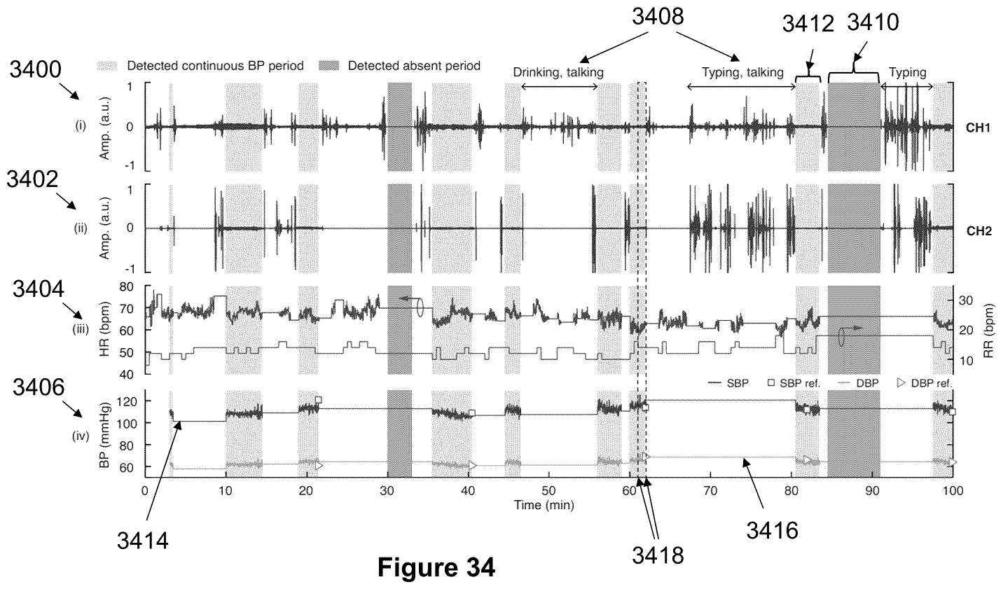

The graphs 3100, 3102 show two 30-second epochs of normalized outputs from both channels CH1 and CH2, while the graphs 3104, 3106 show detected vital signs including heart rate (HR), respiratory rate (RR), systolic blood pressure (SBP) and diastolic blood pressure (DBP). Reference BP measurements are shown for the second epoch of the graph 3106. The graph 3100 as shown in FIG. 31 relates to normalized bandpass filtered signals from 0.1 Hz to 5 Hz, and includes both the time-varying heart phase signal (0.1 Hz-0.8 Hz) component and the time-varying respiration phase signal component (0.9 Hz-5 Hz). The graph 3102 as shown in FIG. 31 relates to the normalized time-varying radial pulse phase signal (i.e. time-varying phase signal from CH2 which has been bandpass filtered at 0.9 Hz-5 Hz). Particularly, an enlarged plot of 2 exemplary epochs at the 61-minute mark of the experiment was shown in FIG. 31. To validate the accuracy of BP measurement, the subject was asked to place his wrist on the CH2 sensor for 2 minutes at approximately 20 minutes apart and the subject's reference BP was measured with a conventional digital BP monitor during these periods. Since only a single pair of SBP and DBP values (i.e. SBP ref. and DBP ref. as shown in graph 3106) can be obtained for each reference measurement, they are considered to be the average values for the entire epoch, as shown in relation to the graph 3106 of FIG. 31 and graph 3406 in relation to FIG. 34. In contrast, within every “continuous BP” epoch, a continuous record of BP values can be provided using the present system without having to actively initiate measurements, making them more insightful and convenient than conventional BP measurements using arm cuffs.

FIG. 34 shows graphs 3400, 3402, 3404, 3406 of long-term health monitoring data for normalized sensor outputs from both channels CH1 and CH2, and the detected vital signs including heart rate (HR), respiratory rate (RR), systolic blood pressure (SBP) and diastolic blood pressure (DBP) in accordance with an embodiment.

FIG. 34 shows the filtered and normalized outputs from both sensor channels as well as all extracted physiological signals for the entire 100-minute working session. The graphs 3400, 3402 show the normalized outputs from CH1 and CH2, respectively, the graph 3404 shows the detected vital signs including heart rate (HR), respiratory rate (RR), and the graph 3406 shows the measured values and the reference values for systolic blood pressure (SBP) and diastolic blood pressure (DBP) of this 100-minute working session. The outputs were pre-processed by dividing them into non-overlapping 30-second epochs, each containing 2 channels (i.e. CH1 and CH2) of data. The graph 3400 as shown in FIG. 34 relates to normalized bandpass filtered signals from 0.1 Hz to 5 Hz, and includes both the time-varying heart phase signal (0.1 Hz-0.8 Hz) component and the time-varying respiration phase signal component (0.9 Hz-5 Hz). The graph 3402 as shown in FIG. 34 relates to the normalized time-varying radial pulse phase signal (i.e. time-varying phase signal from CH2 which has been bandpass filtered at 0.9 Hz-5 Hz). Beat-to-beat HR and RR were calculated for each epoch from the CH1 output. As random movements of the subject caused spikes in the sensor outputs that mask the desired signals, epochs with large movement present, for example during time periods 3408 as shown in FIG. 34, were excluded from the rate calculations. As discussed above, “absent” epochs, in which the subject is not seated on the chair, and “continuous BP” epochs, in which high-quality data is available from both sensor channels for BP calculation can be automatically detected by the system. The “absent” epochs and the “continuous BP epochs” are shown respectively as boxes 3410 and 3412, respectively. PTT was calculated for every “continuous BP” epoch using the LSTM aligner pipeline as previously described in relation to FIG. 25, which was then converted into systolic BP (SBP) values 3414 and diastolic BP (DBP) values 3416 as shown in the graph 3406 using a linear PTT-BP relationship obtained from a calibration procedure prior to the experiment as described in relation to FIGS. 32A and 32B. Also shown in FIG. 34 are the dashed lines 3418 which relate to the two epochs as shown in FIG. 31.

In another embodiment, the textile sensor system of the present disclosure was implemented in an aircraft cabin simulator for demonstrating the SSP metamaterial textile sensor's ability to realise an in-flight ambient monitoring application. Passenger health monitoring during air travel, especially on long-haul flights, is not only important for the early detection of in-flight medical emergencies but also a tremendous tool for gathering information on the wellness and comfort of passengers.

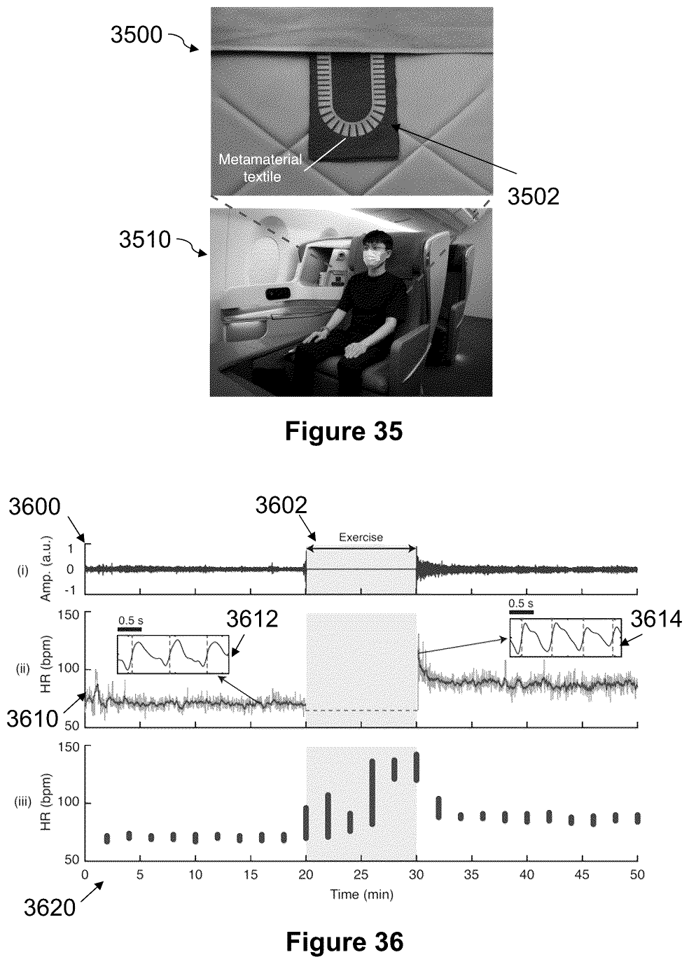

FIG. 35 shows photographs 3500, 3510 of a passenger health monitoring system comprising a metamaterial textile sensor 3502 in accordance with an embodiment. The sensor 3502 was implemented as an in-flight health tracker by integration with an airplane seat. To evaluate the sensing capability, a 50-min continuous HR monitoring trial with a healthy male volunteer was performed.

Particularly, the healthy male volunteer was asked to be seated as shown in the photograph 3510. The volunteer was also asked to wear an Apple Watch (version 7, Apple Inc.) on the left wrist for reference heart rate recording. The evaluation trial lasted for a total of 50 min, comprising 3 consecutive periods. In the first and third 20-min periods, the volunteer was instructed to sit still in the upright position. In the middle 10-min period, the volunteer was instructed to get up from the seat to perform static running exercises. Sensor signals were recorded continuously for the entire duration. Continuous HR using the SSP metamaterial textile sensor 3502 was extracted following the methods as outlined previously. Reference HR from the Apple Watch was obtained from the iOS Health application on a paired iPhone (version 13 Pro, Apple Inc.), which was provided as a minimum-maximum range every 2 min.

FIG. 36 shows graphs 3600, 3610 of normalized output and detected heart rate measured using the passenger health monitoring system of FIG. 35 and a graph 3620 of reference heart rate measured using an Apple® Watch in accordance with an embodiment.

The graph 3600 shows normalized output, where a shaded box 3602 indicates a time period during which the volunteer was instructed to get up from the seat to perform static running exercises. The graph 3600 relates to the normalized time-varying heart phase signal (i.e. time-varying phase signal from CH1 which has been bandpass filtered at 0.9 Hz-5 Hz). The graph 3610 shows detected continuous heart rate measured by the sensor 3502. The insets 3612, 3614 show enlarged plots of the sensor output heart rate waveform before and after performing the static exercises, respectively. Scale bars of 0.5 s are also shown for each of these insets 3612, 3614. The graph 3620 shows reference HR values as measured by the Apple Watch, represented as bars showing the minimum-maximum range for every 2-min period.

FIG. 36 shows the SSP sensor's ability to capture a wide range of HR by increasing the subject's HR by means of exercise. During the seated periods (first and last 20 min), the sensor can collect a continuous beat-to-beat HR of the subject that agrees well with the reference without the volunteer's active involvement. These results therefore show the potential of the SSP metamaterial textile sensors for ambient contactless physiological monitoring in a wide range of complex environments.

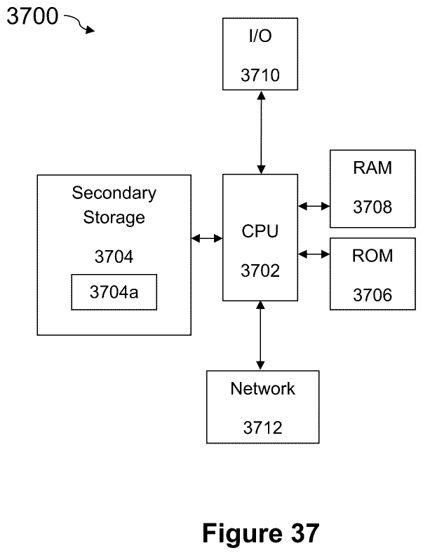

FIG. 37 is a block diagram showing a technical architecture 3700 of the computer 108. The computer 108 includes memory that stores computer program modules which implement methods and/or processes for obtaining vital signs from a body such as a respiratory cycle, heart rate, radial pulse rate and blood pressures.

The technical architecture 3700 of the computer 108 includes a processor 3702 (which may be referred to as a central processor unit or CPU) that is in communication with memory devices including secondary storage 3704 (such as disk drives), read only memory (ROM) 3706, random access memory (RAM) 3708. The processor 3702 may be implemented as one or more CPU chips. The technical architecture 3700 may further comprise input/output (I/O) devices 3710, and network connectivity devices 3712.

The secondary storage 3704 is typically comprised of one or more disk drives or tape drives and is used for non-volatile storage of data and as an over-flow data storage device if RAM 3708 is not large enough to hold all working data. Secondary storage 3704 may be used to store programs which are loaded into RAM 3708 when such programs are selected for execution.

In this embodiment, the secondary storage 3704 has a processing component 3704a comprising non-transitory instructions operative by the processor 3702 to perform various operations of the methods and processes of the present disclosure. These include the various processes described for processing a time-varying phase signal received from the SDR system 106 to obtain a respiratory cycle and beat-to-beat intervals related to the heart rate and the radial pulse rate, and/or for calculating the PTT and obtaining blood pressure values of the body 102 using a machine learning model as described in relation to FIG. 25. In the present embodiment, the secondary storage 3704 also stores the machine learning model comprising the LSTM layers and the fully-connected layers as described. The ROM 3706 is used to store instructions and perhaps data which are read during program execution. The secondary storage 3704, the RAM 3708, and/or the ROM 3706 may be referred to in some contexts as computer readable storage media and/or non-transitory computer readable media.

I/O devices 3710 may include printers, video monitors, liquid crystal displays (LCDs), plasma displays, touch screen displays, keyboards, keypads, switches, dials, mice, track balls, voice recognizers, card readers, paper tape readers, or other well-known input devices.

The network connectivity devices 3712 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identity (RFID), and/or other air interface protocol radio transceiver cards, and other well-known network devices. These network connectivity devices 3712 may enable the processor 3702 to communicate with the Internet or one or more intranets. With such a network connection, it is contemplated that the processor 3702 might receive information from the network, or might output information to the network in the course of performing the above-described method operations. Such information, which is often represented as a sequence of instructions to be executed using processor 3702, may be received from and outputted to the network, for example, in the form of a computer data signal embodied in a carrier wave. Particularly, in the embodiments as described, the network connectivity devices 3712 of the computer 108 are adapted to connect to the SDR system 106 for providing instructions to the SDR system 106 and/or receive outputs (e.g. time-varying phase signals associated with channels 1 and/or 2 of the SDR systems) from the SDR system 106.

The processor 3702 executes instructions, codes, computer programs, scripts which it accesses from hard disk, floppy disk, optical disk (these various disk based systems may all be considered secondary storage 3704), flash drive, ROM 3706, RAM 3708, or the network connectivity devices 3712. While only one processor 3702 is shown, multiple processors may be present. Thus, while instructions may be discussed as executed by a processor, the instructions may be executed simultaneously, serially, or otherwise executed by one or multiple processors.

Although the technical architecture 3700 is described with reference to a computer, it should be appreciated that the technical architecture may be formed by two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the technical architecture 3700 to provide the functionality of a number of servers that is not directly bound to the number of computers in the technical architecture 3700. In an embodiment, the functionality disclosed above may be provided by executing the application and/or applications in a cloud computing environment. Cloud computing may comprise providing computing services via a network connection using dynamically scalable computing resources. A cloud computing environment may be established by an enterprise and/or may be hired on an as-needed basis from a third-party provider.

It is understood that by programming and/or loading executable instructions onto the technical architecture 3700, at least one of the CPU 3702, the RAM 3708, and the ROM 3706 are changed, transforming the technical architecture in part into a specific purpose machine or apparatus having the novel functionality taught by the present disclosure. It is fundamental to the electrical engineering and software engineering arts that functionality that can be implemented by loading executable software into a computer can be converted to a hardware implementation by well-known design rules.

Embodiments of the present disclosure therefore provide one or more thin and conformal metamaterial sensors using conductive textiles that can support efficient spoof-surface-plasmonic propagation at microwave frequencies as localized non-radiating surface waves for sensing vital signs. A multi-channel sensor system was realised that is easily integrated with ordinary common furniture for continuous tracking of multiple vital signs and benchmarking the measured vital signs against gold standard references through evaluation experiments with healthy volunteers. The SSP metamaterial textile sensor's versatile utility in realistic physical environments was also shown through the continuous monitoring demonstrations of respiration rate, heart rate and cuffless blood pressure. The present disclosure thus highlights the potentials of metamaterial textile sensors for pertinent healthcare applications.

The exemplary embodiments of the system and method described above provide an effective way for non-contact sensing of one or more physiological parameters of a body. Further, some advantages associated with the afore described system and method are provided in Table 4 as shown below.

TABLE 4
Advantages of the system or method

| Feature | Benefit/Advantage |
|---|---|
| Sensor based on a class of | Enhanced sensing sensitivity to small |
| thin, flexible waveguides that | physiological motions, such as those |
| can support spoof surface | resulting from the heart and lung actions, |
| plasmons at radio frequencies | thanks to spatial confinement of RF |
| | waves near the sensing target due to the |
| | SSP propagation mode. |
| | Non-radiative RF sensing minimizes |
| | background clutter resulting from random |
| | body motion and multiple objects in the |
| | surrounding environment. |
| | Able to perform multiplexed sensing on |
| | different parts of the body simultaneously |
| | to obtain multiple physiological signals |
| | important for diagnosis of diseases. |
| | Flexibility and robustness of the |
| | metamaterial textile sensors enable the |
| | sensors to be integrated into common |
| | furniture for contactless and continuous |
| | vital signs monitoring. |
| RF based sensing | Does not require physical coupling with |
| methodology | the skin, enabling through-clothing health |
| | monitoring that is convenient and |
| | comfortable for multiple clinical and daily |
| | living settings. |
| Multi-point vital signs sensing | Provide continuous contactless |
| system | monitoring of vital signs, including a |
| | respiration rate, a heart rate and a radial |
| | pulse rate. |
| | Machine learning model used to calculate |
| | blood pressure values based on the |
| | measured heart rate and the measured |
| | radial pulse rate. This provides a cuffless |
| | method for a continuous monitoring of |
| | blood pressure of a body. |

Other advantages may include: (i) the waveguide can be customised by modifying a shape of the metamaterial structure to change the localization of the spoof surface plasmon to modify a sensitivity of the waveguide. For example, the sensitivity of the waveguide can be decreased to increase resilience to environmental impacts or further improved for applications in gesture sensing, proximity detection, and physiological monitoring; and (ii) improved signal security due to the localised sensing range/proximity of the waveguide.

The exemplary embodiment for the system and method as described above is not to be construed as limiting, and variations may be possible. For example, other system, method or software may be used for extracting the detected phase shift between the transmitted signal and the received signal, and/or other system or method can be used for determining the physiological parameter or vital sign and/or to segregate or differentiate between physiological parameters in the measured signals if more than one physiological parameter are captured in the measurements. Further, it should be appreciated that other machine learning models can be trained and used to for aligning the heart signal and the radial pulse signal for use in PTT calculation. Still further, it should be appreciated that other algorithms, other than the Pan-Tompkins algorithm, may be used to extract heart beats or pulse beats.

Other alternative embodiments include: (1) the transmitted signal and the received signal can be provided/received from a same end or a same port to the waveguide (i.e. not limited to transmitting a transmitted signal at one end of the waveguide and receiving a received signal at an opposite end of the waveguide), (2) using other suitable materials (e.g. a metal such as silver or gold, or composites) other than copper on a polyimide substrate or conductive fabrics for the waveguide in propagating at least one spoof surface plasmon mode, (3) using different forms of waveguides (e.g. having repeated C-shaped patterns, repeated rectangular blocks, or repeated cylindrical blocks) other than the comb-shaped pattern, (4) a waveguide structure which does not include the grounding layer or the unpatterned bottom layer, (5) using a radio-frequency transceiver integrated on a chip in place of the SDR system, or other SDR systems including those that have computing functionalities embedded so that they can be operated without a computer, (6) using a carrier signal having a frequency of 400 MHz to 200 GHZ, (7) detecting or sensing other physiological parameters (e.g. a walking pace or a step rate or a posture), (8) digitizing the obtained IQ components or signals using a sampling rate of 0.5 MHz to 10 MHZ, (9) the complex conjugate multiplication of the baseband Tx and Rx signals were low pass filtered and decimated to a frequency in a frequency range of 100 Hz to 500 Hz, (10) sensing a physiological parameter of a body of a human, an animal or a creature, (11) detecting or sensing blood pressure as exemplified above (this can be achieved e.g. by measuring a distal heart rate and a proximal heart rate concurrently, for example by using a separate metamaterial sensor and a synchronized SDR system, and deducing the blood pressure using a pulse transit time associated with the detected distal heart rate and the detected proximal heart rate) which is enabled by the localized sensing modality that allows for real-time concurrent sensing of physiological parameters from multiple locations of the body, (12) using other machine learning model in place of the LSTM network and/or the FC layers, (13) converting PTT to blood pressure values (i.e. systolic and/or diastolic BP values) by using other form of regression and not limited to linear regression (or a linear PTT-BP relationship), for example, a person with chronic heart disease may exhibit a non-linear PTT-BP relationship, (14) different number of matching units N for the metamaterial textile waveguide, for example, 2, 4, 5 or 6, can be used, (15) other shapes of the metamaterial textile sensor (i.e. not limited to U-shape or straight designs) can be used, (16) other placement positions of the metamaterial textile sensors (e.g. placed on a seat of a chair to detect/measure a distal pulse etc.), (17) an application-specific device being engineered to integrate the SDR hardware and computing functionalities so that it can operate on its own, and (18) providing wireless communication functionalities to the application-specific device of (17) so that data can be transmitted wirelessly from the application-specific device to a computer/tablet/display device for display and analysis.

Although only certain embodiments of the present invention have been described in detail, many variations are possible in accordance with the appended claims. For example, features described in relation to one embodiment may be incorporated into one or more other embodiments and vice versa.

1. A sensor for non-contact sensing of a physiological parameter of a body, the sensor comprising:
a waveguide, the waveguide comprises a metamaterial and is configured to receive a transmitted signal and to propagate the transmitted signal in a spoof surface plasmon mode along the waveguide to produce an evanescent electromagnetic field and to provide a received signal,
wherein the waveguide is placed at a predetermined distance away from the body for non-contact sensing of a perturbation produced by a physiological motion of the body using the evanescent electromagnetic field, the perturbation produces a phase shift between the transmitted signal and the received signal for use in determining the physiological parameter of the body.

2. The sensor of claim 1, wherein the waveguide comprises a sensing layer on a sensing side of the waveguide adapted to detect the perturbation produced by the physiological motion of the body, a grounding layer on an opposite side to the sensing side, and a non-electrically conductive layer sandwiched between the sensing layer and the grounding layer, wherein the grounding layer is configured to confine the evanescent electromagnetic field to the sensing side of the waveguide.

3. The sensor of claim 2, wherein the sensing layer comprises a comb-shaped rectangular strip, the comb-shaped rectangular strip having an elongated base and a plurality of teeth extending along and from the elongated base, wherein adjacent teeth of the plurality of teeth is separated by a gap.

4. The sensor of claim 3, wherein a height of the plurality of teeth measured from the elongated base is adapted to vary a degree of wavelength confinement of the spoof surface plasmon mode.

5. A system for non-contact sensing of a physiological parameter of a body, the system comprising one or more sensors according to claim 1, and a software-defined radio (SDR) system configured to provide the transmitted signal and to receive the received signal.

6. The system of claim 5, wherein the SDR system includes a digital-to-analogue converter (DAC), the SDR system is configured to:
generate a digital complex baseband signal;
convert the digital complex baseband signal to form an analogue baseband signal using the DAC;
modulate the analogue baseband signal with a carrier signal to provide the transmitted signal;
demodulate the received signal to obtain in-phase and quadrature (IQ) components associated with the digital complex baseband signal; and
digitise the obtained IQ components.

7. The system of claim 6, wherein the SDR system is configured to perform complex conjugate multiplication of the digital complex baseband signal and the digitised IQ components to determine a phase shift signal associated with the phase shift between the transmitted signal and the received signal.

8. The system of claim 7, wherein the SDR system is configured to filter the phase shift signal with a low-pass filter and to down-sample the filtered phase shift signal to form a decimated phase shift signal.

9. The system of claim 8, wherein the SDR system is adapted to arctangent demodulate and unwrap the decimated phase shift signal to obtain a time-varying phase signal associated with the phase shift between the transmitted signal and the received signal.

10. The system of claim 9, wherein the physiological motion is associated with more than one physiological parameter, the system further comprises a processor and a data storage storing computer program instructions operable to cause the processor to:
process the time-varying phase signal with a bandpass filter to segregate the time-varying phase signal to individual components associated with each of the more than one physiological parameter.

11. The system of claim 9, wherein the one or more sensors includes a first sensor provided at a back of the body adapted to detect a respiration signal and a heart signal associated with the body, and a second sensor provided at a wrist of the body adapted to detect a radial pulse signal associated with the body, the system further comprises a processor and a data storage storing computer program instructions operable to cause the processor to:
process a first time-varying phase signal associated with the first sensor with bandpass filters to segregate the first time-varying phase signal to a time-varying respiration phase signal and a time-varying heart phase signal; and
process a second time-varying phase signal associated with the second sensor with a bandpass filter to obtain a time-varying radial pulse phase signal.

12. The system of claim 11, wherein the data storage further stores computer program instructions operable to cause the processor to:
perform fast Fourier transform on first 15 seconds of each signal segment of the time-varying respiration phase signal to estimate a respiratory period;
calculate a moving-average curve by taking a mean of the time-varying respiration phase signal over a time window equivalent to two times of the respiratory period to generate each data point of the moving-average curve;
calculate intercepts between the moving-average curve and the time-varying respiration phase signal;
identify peaks on the time-varying respiration phase signal using the calculated intercepts, wherein each of the peaks is identified as a maximum between an intercept with a positive slope and an ensuing intercept with a negative slope; and
calculate a respiratory cycle as a time duration between two adjacent peaks.

13. The system of claim 11, wherein the data storage further stores computer program instructions operable to cause the processor to:
calculate a first time derivative waveform for each of the time-varying heart phase signal and the time-varying radial pulse phase signal;
set all negative values of the first time derivative waveform for each of the time-varying heart phase signal and the time-varying radial pulse phase signal to zero to form a resultant waveform for each of the time-varying heart phase signal and the time-varying radial pulse phase signal;
square the resultant waveform associated with each of the time-varying heart phase signal and the time-varying radial pulse phase signal to form a squared signal associated with each of the time-varying heart phase signal and the time-varying radial pulse phase signal;
filter the squared signal using a moving-average filter with a predetermined time window to produce an integrated signal associated with each of the time-varying heart phase signal and the time-varying radial pulse phase signal;
detect peaks in the integrated signal associated with each of the time-varying heart phase signal and the time-varying radial pulse phase signal;
detect peaks in the time-varying heart phase signal and the time-varying radial pulse phase signal;
verify detected peaks in the time-varying heart phase signal and the time-varying radial pulse phase signal using the detected peaks in the integrated signal;
calculate beat locations in the time-varying heart phase signal and the time-varying radial pulse phase signal, each of the beat locations being a nearest preceding positive zero-intercept in relation to each verified peak of the time-varying heart phase signal and the time-varying radial pulse phase signal; and
calculate beat to beat intervals associated with each of the time-varying heart phase signal and the time-varying radial pulse phase signal, the beat to beat intervals being a time interval between successive beat locations, wherein the beat to beat intervals associated with the time-varying heart phase signal relates to a heart rate and the beat to beat intervals associated with the time-varying radial pulse phase signal relates to a radial pulse rate.

14. The system of claim 11, wherein the data storage further stores computer program instructions operable to cause the processor to:
receive the time-varying heart phase signal and the time-varying radial pulse phase signal;
process the time-varying heart phase signal and the time-varying radial pulse phase signal to form a processed time-varying heart phase signal and a processed time-varying radial pulse phase signal;
generate, using a trained machine learning model, an aligned time-varying heart phase signal and an aligned time-varying radial pulse phase signal based on the processed time-varying heart phase signal and the processed time-varying radial pulse phase signal, wherein peaks of the aligned time-varying heart phase signal correspond to electrocardiography (ECG) R-wave peaks and peaks of the aligned time-varying radial pulse phase signal corresponds to photoplethysmography (PPG) maximum first derivative (MFD) points;
calculate a pulse transit time (PTT) as a time delay between one of the peaks of the aligned time-varying heart phase signal and a corresponding one of the peaks of the aligned time-varying radial pulse phase signal; and
convert the calculated PTT to a systolic blood pressure value and a diastolic blood pressure value.

15. The system of claim 14, wherein the data storage further stores computer program instructions operable to cause the processor to:
search, within an ensuing time window of 0.15 s to 0.4 s of the one of the peaks of the aligned time-varying heart phase signal, a local maximum of the aligned time-varying radial pulse phase signal, the local maximum being the corresponding one of the peaks for use in calculating the PTT.

16. The system of claim 14, wherein the data storage further stores computer program instructions operable to cause the processor to:
receive training data comprising training time-varying heart phase signals and training time-varying radial pulse phase signals;
process the training data to form training processed time-varying heart phase signals and training processed time-varying radial pulse phase signals; and
train a machine learning model to form the trained machine learning model, wherein the data storage storing computer program instructions operable to cause the processor to train the machine learning model further stores computer program instructions operable to cause the processor to:
generate, using the machine learning model, training time-varying heart phase signal outputs and training time-varying radial pulse phase signal outputs based on the training processed time-varying heart phase signals and the training processed time-varying radial pulse phase signals; and
minimise, using a regression layer, a mean squared error (MSE) between each of the training time-varying heart phase signal outputs and training time-varying radial pulse phase signal outputs and corresponding target time-varying heart phase signals and time-varying radial pulse phase signals for forming the trained machine learning model.

17. The system of claim 14, wherein the trained machine learning model includes a long short-term memory (LSTM) network followed by a fully connected (FC) layer for each of the time-varying heart phase signal and the time-varying radial pulse phase signal.

18. The system of claim 14, wherein the data storage storing computer program instructions operable to cause the processor to process the time-varying heart phase signal and the time-varying radial pulse phase signal further stores computer program instructions operable to cause the processor to:
left-shift the time-varying heart phase signal by a predetermined amount of time to form the processed time-varying heart phase signal; and
differentiate the time-varying radial pulse phase signal with respect to time to generate a time derivative of the time-varying radial pulse phase signal to form the processed time-varying radial pulse phase signal.

19. The system of claim 14, wherein the data storage further stores computer program instructions operable to cause the processor to:
identify epochs for blood pressure sensing, the identified epochs each being a time window having a predetermined time period during which both the time-varying heart phase signal and the time-varying radial pulse phase signal are present;
calculate a mean heart rate and a mean pulse rate for the identified epochs;
select, one or more candidate epoch among the identified epochs, wherein an absolute difference between the mean heart rate and the mean pulse rate of each of the one or more candidate epoch is less than two beats per minute;
calculate a signal quality metric (Qₑ) for each of the time-varying heart phase signal and the time-varying radial pulse phase signal in each of the one or more candidate epoch as:
$$Qe = \frac{\frac{N}{{t}_{e}}}{\frac{1}{N - 1} {\sum }_{t = 1}^{N - 1} \frac{60}{I ( i )}}$$
where N is a number of detected beats of the time-varying heart phase signal or the time-varying radial pulse phase signal in each of the one or more candidate epoch, tₑ is a length of each of the corresponding one or more candidate epoch in minutes, and I is a beat-to-beat interval from each successive pair of the detected beats in seconds; and
selecting one or more detection epoch from among the one or more candidate epoch for continuous blood pressure detection, wherein the signal quality metric (Qₑ) for each of the time-varying heart phase signal and the time-varying radial pulse phase signal in the one or more detection epoch is more than 0.5.

20. A method for non-contact sensing of a physiological parameter of a body using one or more sensors, wherein each of the one or more sensors comprises a waveguide, the waveguide comprises a metamaterial and is configured to propagate a transmitted signal in a spoof surface plasmon mode along the waveguide to produce an evanescent electromagnetic field and to provide a received signal, the evanescent electromagnetic field being used for non-contact sensing of a perturbation produced by a physiological motion of the body, the method comprising:
(i) placing the waveguide at a predetermined distance away from the body for non-contact sensing of the perturbation;
(ii) providing the transmitted signal to the waveguide;
(iii) receiving the received signal from the waveguide; and
(iv) processing the received signal and the transmitted signal to determine a phase shift between the transmitted signal and the received signal caused by the perturbation for determining the physiological parameter of the body.

21.-30. (canceled)
